(19) United States
(12) Patent Application Publication
(10) Pub. No.: US 2021/0032778 A1
(43) Pub. Date: Feb. 4, 2021

(54) Production Method for Protein Molded Article, Production Method for Protein Solution, and Production Method for Protein

(71) Applicant: Spiber Inc., Tsuruoka-shi, Yamagata (JP)

(72) Inventors: Takuya Saito, Tsuruoka-shi, Yamagata (JP); Ryoji Okada, Tsuruoka-shi, Yamagata (JP); Naoki Matsuzaka, Tsuruoka-shi, Yamagata (JP)

(73) Assignee: Spiber Inc., Tsuruoka-shi, Yamagata (JP)

(21) Appl. No.: 16/966,155

(22) PCT Filed: Jan. 31, 2019
(86) PCT No.: PCT/JP2019/003485

§ 371 Date: Jul. 30, 2020

(30) Foreign Application Priority Data
Jan. 31, 2018 (JP) 2018-015931

(51) Int. Cl. D01F 4/02; C07K 1/113; C07K 14/435

(52) U.S. Cl. CPC D01F 4/02; C07K 14/43518; C07K 1/113

(57) ABSTRACT

The present invention relates to a production method for a protein molded article, including: dissolving a protein in a solvent containing formic acid at a temperature of 40° C. or higher and lower than 80° C. to obtain a protein solution; and molding a protein molded article using the protein solution.

### TECHNICAL FIELD

The present invention relates to a production method for a protein molded article, a production method for a protein solution, and a production method for a protein.

### BACKGROUND ART

A fiber, a film, a porous body, and the like have been conventionally known as molded articles using a protein material as a high molecular material (for example, Patent Literatures 1 to 3). For such a protein molded article, for example, in a case of a fiber, a fiber having excellent physical properties such as strength may be required depending on the purpose of use.

### CITATION LIST

### Patent Literature

[Patent Literature 1] Japanese Patent No. 5540166

[Patent Literature 2] Japanese Patent No. 5678283

[Patent Literature 3] Japanese Patent No. 5796147

### SUMMARY OF INVENTION

### Problems to be Solved by the Invention

An object of the present invention is to provide a production method for a protein molded article, by which a protein molded article having an improved physical property can be easily produced.

### Means for Solving the Problems

The present invention relates to, for example, each of the following inventions.

[1] A production method for a protein molded article, including: dissolving a protein in a solvent containing formic acid at a temperature of 40° C. or higher and lower than 80° C. to obtain a protein solution; and molding a protein molded article using the protein solution.

[2] The production method for a protein molded article according to [1], in which the protein molded article is a protein fiber.

[3] The production method for a protein molded article according to [1] or [2], in which the protein is a structural protein.

[4] The production method for a protein molded article according to [3], in which the structural protein is a spider silk fibroin.

[5] A production method for a protein solution, including dissolving a protein in a solvent containing formic acid at a temperature of 40° C. or higher and lower than 80° C. to obtain a protein solution.

[6] The production method according to [5], in which the protein is a structural protein.

[7] The production method for according to [6], in which the structural protein is a spider silk fibroin.

[8] A production method for a protein, including: dissolving a target protein and impurities in a solvent containing formic acid at a temperature of 40° C. or higher and lower than 80° C. to obtain a protein solution containing the target protein; and treating the protein solution with a poor solvent for the target protein to aggregate the target protein, thereby obtaining the target protein as an aggregate.

### Effects of Invention

According to the present invention, it is possible to provide a production method for a protein molded article, by which a protein molded article having an improved physical property can be easily produced.

According to the production method of the present invention, it is possible to easily produce a protein fiber particularly having improved strength and elongatability among the physical properties, a protein film having a thinner wall while maintaining strength, and a porous body of a protein, having a low apparent density.

### BRIEF DESCRIPTION OF DRAWINGS

FIG. 1 is a schematic diagram illustrating one example of a domain sequence of fibroin.

FIG. 2 is a schematic diagram illustrating one example of a domain sequence of fibroin.

FIG. 3 is a schematic diagram illustrating one example of a domain sequence of fibroin.

FIG. 4 is a graph showing the relationship between the heating temperature and the viscosity of a prepared doping liquid.

FIG. 5 is a graph showing the result of the evaluation of the physical properties of a produced protein fiber.

FIG. 6 is a graph showing the result of the evaluation of the physical properties of a produced protein fiber.

FIG. 7 is a graph showing the result of the GPC measurement of a produced protein fiber.

FIG. 8 is a photograph showing protein solutions prepared using wet bacterial cells containing a spider silk fibroin PRT799.

FIG. 9 is photographs showing the results of SDS-PAGE of proteins purified from wet bacterial cells containing the spider silk fibroin PRT799.

FIG. 10 is a photograph showing protein solutions prepared using dry bacterial cells containing the spider silk fibroin PRT799.

FIG. 11 is photographs showing the results of SDS-PAGE of proteins purified from dry bacterial cells containing the spider silk fibroin PRT799.

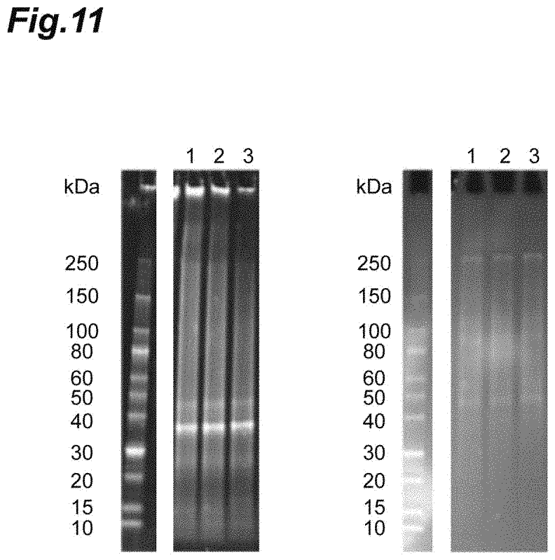

FIG. 12 is photographs showing the results of SDS-PAGE of proteins purified from dry bacterial cells containing the spider silk fibroin PRT799.

FIG. 13 is a photograph showing protein solutions prepared using dry bacterial cells containing a spider silk fibroin PRT918.

FIG. 14 is photographs showing the results of SDS-PAGE of proteins purified from dry bacterial cells containing the spider silk fibroin PRT918.

### EMBODIMENTS FOR CARRYING OUT THE INVENTION

Hereinafter, embodiments of the present invention will be described in detail. However, the present invention is not limited to the following embodiments.

[Production Method for Protein Molded Article]

A production method for a protein molded article of the present embodiment includes dissolving (dissolving process) a protein in a solvent containing formic acid at a temperature of 40° C. or higher and lower than 80° C. to obtain a protein solution and molding (molding process) a protein molded article using the protein solution.

According to the production method for a protein molded article of the present embodiment, it is possible to produce a protein molded article having improved physical properties. A protein solution obtained in the dissolving process, in which gelation is suppressed, is suitable as a doping liquid in a case of molding a protein fiber.

The reason why a protein molded article having improved physical properties can be obtained by the production method of the present embodiment is not clear, but the inventors of the present invention speculate as follows. First, by dissolving a protein in a solvent containing formic acid at a temperature of 40° C. or higher and lower than 80° C., a part of the protein is decomposed and decomposed products having lower molecular weights increase. It is speculated that the lower molecular weights unexpectedly contribute to the strength and the like, and as a result, a protein molded article having improved physical properties can be obtained.

(Protein)

The type of protein is not particularly limited and may be, for example, a structural protein. The structural protein refers to a protein forming a biological structure or a protein derived a biological structure. That is, the structural protein may be a naturally occurring structural protein and a modified protein in which a part of the amino acid sequence (for example, 10% or less of the amino acid sequence) is modified depending on the amino acid sequence of the naturally occurring structural protein.

Examples of the structural protein include fibroin, collagen, resilin, elastin, and keratin, and proteins derived therefrom. The fibroin may be, for example, one or more selected from the group consisting of a silk fibroin, a spider silk fibroin, and a hornet silk fibroin. The structural protein may be a silk fibroin or a spider silk fibroin, or a combination thereof.

The fibroin according to the present embodiment includes a naturally occurring fibroin and a modified fibroin. In the present specification, the “naturally occurring fibroin” means a fibroin having the same amino acid sequence as the naturally occurring fibroin, and the “modified fibroin” means a fibroin having an amino acid sequence different from that of the naturally occurring fibroin.

The fibroin according to the present embodiment is preferably a spider silk fibroin. The spider silk fibroin includes a natural spider silk fibroin and a modified fibroin derived from the natural spider silk fibroin. Specific examples of the natural spider silk fibroin include a spider silk protein produced by spiders.

The fibroin according to the present embodiment may be, for example, a protein including a domain sequence represented by Formula 1: [(A)ₙ motif−REP]ₘ or Formula 2: [(A)ₙ motif−REP]ₘ−(A)ₙ motif. In the fibroin according to the present embodiment, an amino acid sequence (an N-terminal sequence and a C-terminal sequence) may be further added to either or both of the N-terminal side and the C-terminal side of the domain sequence. The N-terminal sequence and the C-terminal sequence, although not limited thereto, are typically regions that do not have repetitions of amino acid motifs characteristic of fibroin and consist of amino acids of about 100 residues.

The term “domain sequence” as used herein refers to an amino acid sequence which produces a crystalline region (typically, corresponds to (A)ₙ motif of an amino acid sequence) and an amorphous region (typically, corresponds to REP of an amino acid sequence) peculiar to fibroin and means an amino acid sequence represented by Formula 1: [(A)ₙ motif−REP]ₘ or Formula 2: [(A)ₙ motif−REP]ₘ−(A)ₙ motif. Here, the (A)ₙ motif represents an amino acid sequence mainly including alanine residues, and the number of amino acid residues in the (A)ₙ motif is 2 to 27. The number of amino acid residues in the (A)ₙ motif may be an integer of 2 to 20, 4 to 27, 4 to 20, 8 to 20, 10 to 20, 4 to 16, 8 to 16, or 10 to 16. Further, the proportion of the number of alanine residues with respect to the total number of amino acid residues in the (A)ₙ motif may be 40% or more, 60% or more, 70% or more, 80% or more, 83% or more, 85% or more, 86% or more, 90% or more, 95% or more, or 100% (meaning that the (A)ₙ motif is composed of only alanine residues). In a plurality of (A)ₙ motifs present in the domain sequence, at least seven of the (A)ₙ motif may be composed of only alanine residues. REP represents an amino acid sequence composed of 2 to 200 amino acid residues. The REP may represent an amino acid sequence composed of 10 to 200 amino acid residues. m represents an integer of 2 to 300 and may be an integer of 10 to 300. The plurality of (A)ₙ motifs may have the same amino acid sequence or amino acid sequences different from each other. The plurality of REPs may have the same amino acid sequence or amino acid sequences different from each other.

Examples of the naturally occurring fibroin include a protein including a domain sequence represented by Formula 1: [(A)ₙ motif−REP]ₘ or Formula 2: [(A)ₙ motif−REP]ₘ−(A)ₙ motif. Specific examples of the naturally occurring fibroin include a fibroin produced by insects or spiders.

Examples of the fibroin produced by insects include silk proteins produced by silkworms such as Bombyx mori, Bombyx mandarina, Antheraea yamamai, Anteraea pernyi, Eriogyna pyretorum, Pilosamia Cynthia ricini, Samia cynthia, Caligura japonica, Antheraea mylitta, and Antheraea assama; and hornet silk proteins discharged by larvae of Vespa simillima xanthoptera.

A more specific example of the fibroin produced by insects includes a silkworm fibroin L chain (GenBank Accession No. M76430 (base sequence) and AAA27840.1 (amino acid sequence)).

Examples of the fibroin produced by spiders include spider silk proteins produced by spiders belonging to the genus Araneus such as Araneus ventricosus, Araneus diadematus, Araneus pinguis, Araneus pentagrammicus and Araneus nojimai, spiders belonging to the genus Neoscona such as Neoscona scylla, Neoscona nautica, Neoscona adianta and Neoscona scylloides, spiders belonging to the genus Pronus such as Pronous minutes, spiders belonging to the genus Cyrtarachne such as Cyrtarachne bufo and Cyrtarachne inaequalis, spiders belonging to the genus Gasteracantha such as Gasteracantha kuhli and Gasteracantha mammosa, spiders belonging to the genus Ordgarius such as Ordgarius hobsoni and Ordgarius sexspinosus, spiders belonging to the genus Argiope such as Argiope amoena, Argiope minuta and Argiope bruennich, spiders belonging to the genus Arachnura such as Arachnura logio, spiders belonging to the genus Acusilas such as Acusilas coccineus, spiders belonging to the genus Cytophora such as Cyrtophora moluccensis, Cyrtophora exanthematica and Cyrtophora unicolor, spiders belonging to the genus Poltys such as Poltys illepidus, spiders belonging to the genus Cyclosa such as Cyclosa octotuberculata, Cyclosa sedeculata, Cyclosa vallata and Cyclosa atrata, and spiders belonging to the genus Chorizopes such as Chorizopes nipponicus; and spider silk proteins produced by spiders belonging to the genus Tetragnatha such as Tetragnatha praedonia, Tetragnatha maxillosa, Tetragnatha extensa and Tetragnatha squamata, spiders belonging to the genus Leucauge such as Leucauge magnifica, Leucauge blanda and Leucauge subblanda, spiders belonging to the genus Nephila such as Nephila clavata and Nephila pilipes, spiders belonging to the genus Menosira such as Menosira ornata, spiders belonging to the genus Dyschiriognatha such as Dyschiriognatha tenera, spiders belonging to the genus Latrodectus such as Latrodectus mactans, Latrodectus hasseltii, Latrodectus geometricus and Latrodectus tredecimguttatus, and spiders belonging to the family Tetragnathidae such as spiders belonging to the genus Euprosthenops. Examples of spider silk proteins include traction yarn proteins such as MaSp (MaSp1 and MaSp2) and ADF (ADF3 and ADF4), and MiSp (MiSp1 and MiSp2).

More specific examples of the spider silk protein produced by spiders include fibroin-3 (adf-3) [derived from Araneus diadematus] (GenBank Accession No. AAC47010 (amino acid sequence), U47855 (base sequence)), fibroin-4 (adf-4) [derived from Araneus diadematus] (GenBank Accession No. AAC47011 (amino acid sequence), U47856 (base sequence)), dragline silk protein spidroin 1 [derived from Nephila clavipes] (GenBank Accession No. AAC04504 (amino acid sequence), U37520 (base sequence)), major ampulate spidroin 1 [derived from Latrodectus hesperus] (GenBank Accession No. ABR68856 (amino acid sequence), EF595246 (base sequence)), dragline silk protein spidroin 2 [derived from Nephila clavata] (GenBank Accession No. AAL32472 (amino acid sequence), AF441245 (base sequence)), major ampulate spidroin 1 [derived from Euprosthenops australis] (GenBank Accession No. CAJ00428 (amino acid sequence), AJ973155 (base sequence)) and major ampullate spidroin 2 [Euprosthenops australis] (GenBank Accession No. CAM32249.1 (amino acid sequence), AM490169 (base sequence)), minor ampullate silk protein 1 [Nephila clavipes] (GenBank Accession No. AAC14589.1 (amino acid sequence), minor ampullate silk protein 2 [Nephila clavipes] (GenBank Accession No. AAC14591.1 (amino acid sequence)), and minor ampullate spidroin-like protein [Nephilengys cruentata] (GenBank Accession No. ABR37278.1 (amino acid sequence)).

As a further specified example of the naturally occurring fibroin, a fibroin whose sequence information is registered in NCBI GenBank may be mentioned. For example, sequences thereof may be confirmed by extracting sequences in which spidroin, ampullate, fibroin, “silk and polypeptide”, or “silk and protein” is described as a keyword in DEFINITION among sequences including INV as DIVISION in sequence information registered in NCBI GenBank, sequences in which a specific character string of a product is described from CDS or sequences in which a specific character string is described from SOURCE to TISSUE TYPE.

(Modified Fibroin)

The modified fibroin may be, for example, a fibroin whose amino acid sequence has been modified depending on the amino acid sequence of the naturally occurring fibroin (for example, a fibroin whose amino acid sequence has been modified by altering a cloned gene sequence of naturally occurring fibroin) or a fibroin artificially designed and synthesized independently of naturally occurring fibroin (for example, a fibroin having a desired amino acid sequence by chemically synthesizing a nucleic acid encoding the designed amino acid sequence).

The modified fibroin can be obtained, for example, by carrying out the modification of an amino acid sequence equivalent to the substitution, deletion, insertion and/or addition of one or a plurality of amino acid residues with respect to, for example, a cloned gene sequence of a naturally occurring fibroin. The substitution, deletion, insertion, and/or addition of an amino acid residue may be carried out by methods well known to those skilled in the art, such as site-directed mutagenesis. Specifically, the modifications may be carried out by methods described in literature such as Nucleic Acid Res. 10, 6487 (1982) and Methods in Enzymology, 100, 448 (1983).

The modified fibroin may be, for example, a modified fibroin derived from a silk protein produced by a silkworm or a modified fibroin derived from a spider silk protein produced by spiders.

Specific examples of the modified fibroin include: a modified fibroin (first modified fibroin) derived from a large spinal canal bookmark silk protein produced in a major ampullate of a spider; a modified fibroin (second modified fibroin) having a reduced content of glycine residue; a modified fibroin (third modified fibroin) with a reduced content of (A)ₙ motif; a modified fibroin (fourth modified fibroin) with a reduced content of glycine residue and a reduced content of (A)ₙ motif; a modified fibroin (fifth modified fibroin) having a domain sequence including a region locally having a high hydropathy index; and a modified fibroin (sixth modified fibroin) having a domain sequence with a reduced content of glutamine residue.

As a modified fibroin (first modified fibroin) derived from a large spinal canal bookmark silk protein produced in the major ampullate gland of a spider, a protein including a domain sequence represented by Formula 1: [(A)ₙ motif−REP]ₘ is mentioned. In the first modified fibroin, n in Formula 1 is preferably an integer of 3 to 20, more preferably an integer of 4 to 20, still more preferably an integer of 8 to 20, even more preferably an integer of 10 to 20, even further more preferably an integer of 4 to 16, particularly preferably an integer of 8 to 16, and most preferably an integer of 10 to 16. In the first modified fibroin, the number of amino acid residues constituting REP in Formula 1 is preferably 10 to 200 residues, more preferably 10 to 150 residues, and still more preferably 20 to 100 residues, and even more preferably 20 to 75 residues. In the first modified fibroin, the total number of glycine residues, serine residues, and alanine residues contained in the amino acid sequence represented by Formula 1: [(A)ₙ motif−REP]ₘ is preferably 40% or more, more preferably 60% or more, and still more preferably 70% or more with respect to the total number of amino acid residues.

The first modified fibroin may be a polypeptide including an amino acid sequence unit represented by Formula 1: [(A)ₙ motif−REP]ₘ, and having a C-terminal sequence which is the amino acid sequence set forth in any of SEQ ID NOs: 1 to 3 or an amino acid sequence having 90% or more homology with the amino acid sequence set forth in any of SEQ ID NOs: 1 to 3.

The amino acid sequence set forth in SEQ ID NO: 1 is identical to the amino acid sequence consisting of 50 amino acid residues at the C-terminal of the amino acid sequence of ADF3 (GI: 1263287, NCBI). The amino acid sequence set forth in SEQ ID NO: 2 is identical to the amino acid sequence obtained by removing 20 residues from the C-terminal of the amino acid sequence set forth in SEQ ID NO: 1. The amino acid sequence set forth in SEQ ID NO: 3 is identical to the amino acid sequence obtained by removing 29 residues from the C-terminal of the amino acid sequence set forth in SEQ ID NO: 1.

More specific examples of the first modified fibroin include a modified fibroin including (1-i) the amino acid sequence set forth in SEQ ID NO: 4 or (1-ii) the amino acid sequence having 90% or more sequence identity with the amino acid sequence set forth in SEQ ID NO: 4. The sequence identity is preferably 95% or more.

The amino acid sequence set forth in SEQ ID NO: 4 is an amino acid sequence obtained by approximately doubling repeating regions from the first repeating region to the 13th repeating region and performing mutation so that translation is terminated at the 1154th amino acid residue in an amino acid sequence obtained by adding the amino acid sequence (SEQ ID NO: 5) consisting of a start codon, a His10 tag, and a recognition site for HRV3C protease (human rhinovirus 3C protease) to the N-terminal of ADF3. The C-terminal amino acid sequence of the amino acid sequence set forth in SEQ ID NO: 4 is identical to the amino acid sequence set forth in SEQ ID NO: 3.

The modified fibroin of (1-i) may consist of the amino acid sequence set forth in SEQ ID NO: 4.

A domain sequence of a modified fibroin (second modified fibroin) having a reduced content of the glycine residue has an amino acid sequence with a reduced content of the glycine residue, as compared with a naturally occurring fibroin. It can be said that the second modified fibroin has an amino acid sequence equivalent to an amino acid sequence in which at least one or a plurality of glycine residues in REP are substituted with other amino acid residues, as compared with naturally occurring fibroin.

The domain sequence of the second modified fibroin may have an amino acid sequence equivalent to an amino acid sequence in which one glycine residue in at least one or the plurality of motif sequences, at least one of which is selected from GGX and GPGXX (where G represents a glycine residue, P represents a proline residue, and X represents an amino acid residue other than glycine) in REP, is substituted with other amino acid residue, as compared with naturally occurring fibroin.

In the second modified fibroin, the proportion of the motif sequences in which the above-described glycine residue is substituted with other amino acid residue may be 10% or more with respect to the entire motif sequences.

The second modified fibroin may include a domain sequence represented by Formula 1: [(A)ₙ motif−REP]ₘ and have an amino acid sequence in which z/w is 30% or more, 40% or more, 50% or more, or 50.9% or more, in a case where the total number of amino acid residues consisting of XGX (where X represents an amino acid residue other than glycine) included in all REPs in a sequence excluding the sequence from the (A)ₙ motif located at the most C-terminal side to the C-terminal of the domain sequence from the domain sequence is denoted by z, and the total number of amino acid residues in the sequence excluding the sequence from the (A)ₙ motif located at the most C-terminal side to the C-terminal of the domain sequence from the domain sequence is denoted by w. The number of alanine residues is 83% or more with respect to the total number of amino acid residues in the (A)ₙ motif, preferably 86% or more, more preferably 90% or more, still more preferably 95% or more, and even still more preferably 100% (which means that the (A)ₙ motif consists of only alanine residues).

In the second modified fibroin, the content proportion of an amino acid sequence consisting of XGX is preferably increased by substituting one glycine residue in GGX motif with other amino acid residue. In the second modified fibroin, the content proportion of an amino acid sequence consisting of GGX in the domain sequence is preferably 30% or less, more preferably 20% or less, still more preferably 10% or less, even still more preferably 6% or less, still further preferably 4% or less, and particularly preferably 2% or less. The content proportion of an amino acid sequence consisting of GGX in a domain sequence can be calculated by the same method as the following method for calculating the content proportion (z/w) of the amino acid sequence consisting of XGX.

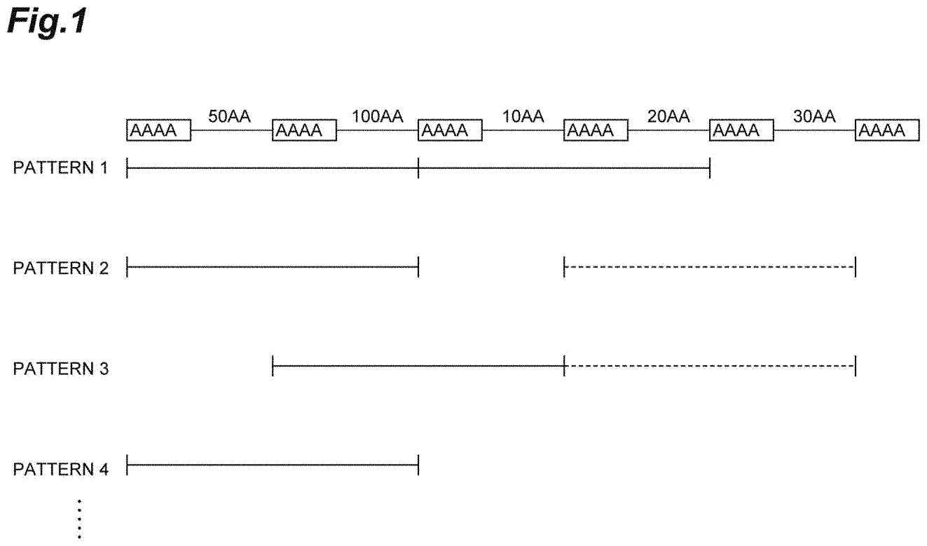

The calculation method for z/w will be described in more detail. First, in a fibroin (a modified fibroin or a naturally occurring fibroin) represented by Formula 1: ([(A)ₙ motif−REP]ₘ−(A)ₙ motif], the amino acid sequence consisting of XGX is extracted from all REPs included in a sequence excluding a sequence from the (A)ₙ motif located closest to the C-terminal side to the C-terminal of the domain sequence from the domain sequence. The total number of amino acid residues constituting XGX is z. For example, in a case where 50 amino acid sequences consisting of XGX (without overlap) are extracted, z is 50×3=150. Further, for example, in a case where there exists an X (a central X) contained in two XGXs, as in the case of an amino acid sequence consisting of XGXGX, the calculation is performed by subtracting the overlapping portion (in the case of XGXGX, it is counted as 5 amino acid residues). w is the total number of amino acid residues included in the sequence excluding a sequence from the (A)ₙ motif located closest to the C terminus to the C terminus of the domain sequence from the domain sequence. For example, in the case of the domain sequence illustrated in FIG. 1, w is 4+50+4+100+4+10+4+20+4+30=230 (the (A)ₙ motif located closest to the C-terminal side is excluded.). Next, z/w (%) can be calculated by dividing z by w.

In the second modified fibroin, z/w is preferably 50.9% or more, more preferably 56.1% or more, still more preferably 58.7% or more, even still more preferably 70% or more, and still further preferably 80% or more. The upper limit of z/w is not particularly limited, but, for example, it may be 95% or less.

The second modified fibroin can be obtained by, for example, modifying a cloned naturally occurring fibroin gene sequence such that at least a part of a base sequence encoding a glycine residue is substituted with other amino acid residue to encode other amino acid residue. In this case, one glycine residue in GGX motif and GPGXX motif may be selected as the glycine residue to be modified or may be substituted so that z/w is 50.9% or more. Alternatively, a modified fibroin may also be obtained, for example, by designing an amino acid sequence satisfying the above-described aspect based on the amino acid sequence of a naturally occurring fibroin and chemically synthesizing a nucleic acid encoding the designed amino acid sequence. In any case, with respect to the amino acid sequence of a naturally occurring fibroin, in addition to the modification corresponding to the substitution of the glycine residue in REP with other amino acid residue, further modification of amino acid sequence corresponding to substitution, deletion, insertion and/or addition of one or a plurality of amino acid residues may be carried out.

The other amino acid residue described above is not particularly limited as long as it is an amino acid residue other than glycine residue, but it is preferably a hydrophobic amino acid residue such as valine (V) residue, leucine (L) residue, isoleucine (I) residue, methionine (M) residue, proline (P) residue, phenylalanine (F) residue, and tryptophan (W) residue, or a hydrophilic amino acid residues such glutamine (Q) residue, asparagine (N) residue, serine (S) residue, lysine (K) residue, and glutamic acid (E) residue, more preferably valine (V) residue, leucine (L) residue, isoleucine (I) residue, and glutamine (Q) residue, and still more preferably glutamine (Q) residue.

A more specific example of the second modified fibroin may be a modified fibroin including (2-i) the amino acid sequence set forth in SEQ ID NO: 6, SEQ ID NO: 7, SEQ ID NO: 8, or SEQ ID NO: 9, or (2-ii) an amino acid sequence having 90% or more sequence identity with the amino acid sequence set forth in SEQ ID NO: 6, SEQ ID NO: 7, SEQ ID NO: 8, or SEQ ID NO: 9.

The modified fibroin of (2-i) will be described. The amino acid sequence set forth in SEQ ID NO: 6 is obtained by substituting all GGXs in REP of the amino acid sequence set forth in SEQ ID NO: 10 equivalent to a naturally occurring fibroin with GQX. The amino acid sequence set forth in SEQ ID NO: 7 is obtained by deleting one of every two (A)ₙ motifs from the N-terminal side to the C-terminal side in the amino acid sequence set forth in SEQ ID NO: 6 and further inserting one [(A)ₙ motif−REP] just before the C-terminal sequence. The amino acid sequence set forth in SEQ ID NO: 8 is obtained by inserting two alanine residues at the C-terminal side of each (A)ₙ motif of the amino acid sequence set forth in SEQ ID NO: 7, and further substituting a part of glutamine (Q) residues with serine (S) residues and deleting a part of amino acids on the N-terminal side so that the molecular weight thereof is approximately the same as that of SEQ ID NO: 7. The amino acid sequence set forth in SEQ ID NO: 9 is an amino acid sequence obtained by adding a His tag to the C-terminal of a sequence obtained by repeating, four times, a region of 20 domain sequences (where several amino acid residues on the C-terminal side of the region are substituted) present in the amino acid sequence set forth in SEQ ID NO: 11.

The value of z/w in the amino acid sequence set forth SEQ ID NO: 10 (corresponds to a naturally occurring fibroin) is 46.8%. The values of z/w in the amino acid sequences set forth in SEQ ID NO: 6, the amino acid sequence set forth in SEQ ID NO: 7, the amino acid sequence set forth in SEQ ID NO: 8, and the amino acid sequence set forth in SEQ ID NO: 9 are respectively 58.7%, 70.1%, 66.1%, and 70.0%. In addition, the values of x/y with a Giza ratio (described later) of 1:1.8 to 11.3 in the amino acid sequences set forth in SEQ ID NO: 10, SEQ ID NO: 6, SEQ ID NO: 7, SEQ ID NO: 8, and SEQ ID NO: 9 are respectively 15.0%, 15.0%, 93.4%, 92.7%, and 89.3%.

The modified fibroin of (2-i) may consist of the amino acid sequence set forth in SEQ ID NO: 6, SEQ ID NO: 7, SEQ ID NO: 8, or SEQ ID NO: 9.

The modified fibroin of (2-ii) includes an amino acid sequence having 90% or more sequence identity with the amino acid sequence set forth in SEQ ID NO: 6, SEQ ID NO: 7, SEQ ID NO: 8, or SEQ ID NO: 9. The modified fibroin of (2-ii) is also a protein including a domain sequence represented by Formula 1: [(A)ₙ motif−REP]ₘ. The sequence identity is preferably 95% or more.

The modified fibroin of (2-ii) preferably has 90% or more sequence identity with the amino acid sequence set forth in SEQ ID NO: 6, SEQ ID NO: 7, SEQ ID NO: 8, or SEQ ID NO: 9, and in a case where the total number of amino acid residues in the amino acid sequence consisting of XGX (where X represents an amino acid residue other than glycine) included in REP is z, and the total number of amino acid residues in REP in the domain sequence is w, z/w is preferably 50.9% or more.

The second modified fibroin may include a tag sequence at either or both of the N-terminal and C-terminal. This makes it possible to isolate, immobilize, detect, and visualize the modified fibroin.

The tag sequence may be, for example, an affinity tag utilizing specific affinity (binding property, affinity) with another molecule. As a specific example of the affinity tag, a histidine tag (a His tag) can be mentioned. The His tag is a short peptide in which about 4 to 10 histidine residues are arranged and has a property of specifically binding to a metal ion such as nickel, and thus it can be used for isolation of a modified fibroin by a chelating metal chromatography. A specific example of the tag sequence may include the amino acid sequence set forth in SEQ ID NO: 12 (amino acid sequence including a His tag and a hinge sequence).

In addition, a tag sequence such as glutathione-S-transferase (GST) that specifically binds to glutathione or a maltose binding protein (MBP) that specifically binds to maltose can also be used.

Further, an “epitope tag” utilizing an antigen-antibody reaction can also be used. By adding a peptide (an epitope) showing antigenicity as a tag sequence, an antibody against the epitope can be bound. Examples of the epitope tag include an HA (peptide sequence of hemagglutinin of influenza virus) tag, a myc tag, and a FLAG tag. The modified fibroin can be easily purified with high specificity by utilizing an epitope tag.

It is also possible to use a tag sequence which can be cleaved with a specific protease. By treating a protein adsorbed via the tag sequence with a protease, it is also possible to recover a modified fibroin cleaved from the tag sequence.

A more specific example of the second modified fibroin including a tag sequence may be a modified fibroin including (2-iii) the amino acid sequence set forth in SEQ ID NO: 13, SEQ ID NO: 11, SEQ ID NO: 14, or SEQ ID NO: 15, or (2-iv) an amino acid sequence having 90% or more sequence identity with the amino acid sequence set forth in SEQ ID NO: 13, SEQ ID NO: 11, SEQ ID NO: 14, or SEQ ID NO: 15.

The amino acid sequences set forth in SEQ ID NO: 16, SEQ ID NO: 17, SEQ ID NO: 13, SEQ ID NO:11, SEQ ID NO: 14, and SEQ ID NO: 15 are respectively amino acid sequences obtained by adding the amino acid sequence (including a His tag and a hinge sequence) set forth in SEQ ID NO: 12 to the N-terminal of the amino acid sequences set forth in SEQ ID NO: 10, SEQ ID NO: 18, SEQ ID NO: 6, SEQ ID NO: 7, SEQ ID NO: 8, and SEQ ID NO: 9.

The modified fibroin of (2-iii) may consist of the amino acid sequence set forth in SEQ ID NO: 13, SEQ ID NO: 11, SEQ ID NO: 14, or SEQ ID NO: 15.

The modified fibroin of (2-iv) includes an amino acid sequence having 90% or more sequence identity with the amino acid sequence set forth in SEQ ID NO: 13, SEQ ID NO: 11, SEQ ID NO: 14, or SEQ ID NO: 15. The modified fibroin of (2-iv) is also a protein including a domain sequence represented by Formula 1: [(A)ₙ motif−REP]ₘ. The sequence identity is preferably 95% or more.

The modified fibroin of (2-iv) preferably has 90% or more sequence identity with the amino acid sequence set forth in SEQ ID NO: 13, SEQ ID NO: 11, SEQ ID NO: 14, or SEQ ID NO: 15, and in a case where the total number of amino acid residues in the amino acid sequence consisting of XGX (where X represents an amino acid residue other than glycine) included in REP is z, and the total number of amino acid residues in REP in the domain sequence is w, z/w is preferably 50.9% or more.

The second modified fibroin may include a secretory signal for releasing the protein produced in the recombinant protein production system to the outside of a host. The sequence of the secretory signal can be appropriately set depending on the type of the host.

A domain sequence of a modified fibroin (third modified fibroin) having a reduced content of the (A)ₙ motif has an amino acid sequence with a reduced content of the (A)ₙ motif, as compared with a naturally occurring fibroin. It can be said that the domain sequence of the third modified fibroin has an amino acid sequence equivalent to an amino acid sequence in which at least one or a plurality of (A)ₙ motifs are deleted, as compared with naturally occurring fibroin.

The third modified fibroin may have an amino acid sequence equivalent to an amino acid sequence in which 10% to 40% of (A)ₙ motifs are deleted from naturally occurring fibroin.

The domain sequence of the third modified fibroin may have an amino acid sequence equivalent to an amino acid sequence obtained by deleting at least one of every one to three (A)ₙ motifs from the N-terminal side to the C-terminal side, as compared with naturally occurring fibroin.

The domain sequence of the third modified fibroin may have an amino acid sequence equivalent to an amino acid sequence obtained by repeating deletion of at least two consecutive (A)ₙ motifs and deletion of one (A)ₙ motif in this order from the N-terminal side to the C-terminal side, as compared with naturally occurring fibroin.

The domain sequence of the third modified fibroin may have an amino acid sequence equivalent to an amino acid sequence obtained by deleting at least one of every two (A)ₙ motifs from the N-terminal side to the C-terminal side.

The third modified fibroin may include a domain sequence represented by Formula 1: [(A)ₙ motif−REP]ₘ, and may have an amino acid sequence in which x/y is 20% or more, 30% or more, 40% or more, or 50% or more, in a case where the number of amino acid residues of two [(A)ₙ motif−REP] units adjacent to each other is sequentially compared from the N-terminal side to the C-terminal side and then the number of amino acid residues of one REP having a small number of amino acid residues is set to 1, the maximum total value of the added numbers of amino acid residues of two [(A)ₙ motif−REP] units adjacent to each other, in which the ratio of the number of amino acid residues of the other REP is 1.8 to 11.3, is denoted by x, and the total number of amino acid residues in the domain sequence is denoted by y. The number of alanine residues is 83% or more with respect to the total number of amino acid residues in the (A)ₙ motif, preferably 86% or more, more preferably 90% or more, still more preferably 95% or more, and even still more preferably 100% (which means that the (A)ₙ motif consists of only alanine residues).

The method for calculating x/y will be described in more detail with reference to FIG. 1. FIG. 1 illustrates a domain sequence obtained by removing an N-terminal sequence and a C-terminal sequence from fibroin. The domain sequence has a sequence of, from the N-terminal side (left side), (A)ₙ motif−first REP (50 amino acid residues)−(A)ₙ motif−second REP (100 amino acid residues)−(A)ₙ motif−third REP (10 amino acid residues)−(A)ₙ motif−fourth REP (20 amino acid residues)−(A)ₙ motif−fifth REP (30 amino acid residues)−(A)ₙ motif sequence.

Two [(A)ₙ motif−REP] units adjacent to each other are sequentially selected from the N-terminal side toward the C-terminal side so that the units are not overlapped with each other. In this case, an unselected [(A)ₙ motif−REP] unit may be present. In FIG. 1, pattern 1 (comparison of first REP and second REP, and comparison of third REP and fourth REP), pattern 2 (comparison of first REP and second REP, and comparison of fourth REP and fifth REP), pattern 3 (comparison of second REP and third REP, and comparison of fourth REP and fifth REP), and pattern 4 (comparison of first REP and second REP). There are other selection methods other than these methods.

Subsequently, for each pattern, the number of amino acid residues of each REP in two selected [(A)ₙ motif−REP] units adjacent to each other is compared. The comparison is performed by determining the ratio of the number of amino acid residues of one REP to the number of amino acid residues of the other REP having the smaller number of amino acid residues so that the number of amino acid residues in the other REP is set to 1. For example, in the case of comparing the first REP (50 amino acid residues) and the second REP (100 amino acid residues), when the first REP having the smaller number of amino acid residues is set to 1, the ratio of the number of amino acid residues of the second REP is 100/50=2. Similarly, in the case of comparing the fourth REP (20 amino acid residues) and the fifth REP (30 amino acid residues), when the fourth REP having the smaller number of amino acid residues is set to 1, the ratio of the number of amino acid residues of the fifth REP is 30/20=1.5.

In FIG. 1, in a case where one group of [(A)ₙ motif−REP] units having the smaller number of amino acid residues is set to 1, the other group in which the ratio of the number of amino acid residues is 1.8 to 11.3 is indicated by a solid line. Hereinafter, this ratio is referred to as a Giza ratio. In a case where one group of [(A)ₙ motif−REP] units having the smaller number of amino acid residues is set to 1, the other group in which the ratio of the number of amino acid residues is less than 1.8 or more than 11.3 is indicated by a broken line.

In each pattern, the total numbers of amino acid residues of two [(A)ₙ motif−REP] units adjacent to each other indicated by solid lines are added (not only the number of REPs but also the number of the amino acid residues in the (A)ₙ motif are added.) Then, the added total values are compared, and the total value (maximum value of the total values) of the pattern having the maximum total value is denoted by x. In the example illustrated in FIG. 1, the total value of the pattern 1 is the maximum.

Next, x/y (%) can be calculated by dividing x by y which is the total number of the amino acid residues of the domain sequence.

In the third modified fibroin, x/y is preferably 50% or more, more preferably 60% or more, still more preferably 65% or more, even still more preferably 70% or more, still further preferably 75% or more, and particularly preferably 80% or more. The upper limit of x/y is not particularly limited, but for example, it may be 100% or less. In a case where the Giza ratio is 1:1.9 to 11.3, x/y is preferably 89.6% or more. In a case where the Giza ratio is 1:1.8 to 3.4, x/y is more preferably 77.1% or more. In a case where the Giza ratio is 1:1.9 to 8.4, x/y is still more preferably 75.9% or more. In a case where the Giza ratio is 1:1.9 to 4.1, x/y is even still more preferably 64.2% or more.

In a case where the third modified fibroin is a modified fibroin in which at least seven (A)ₙ motifs present in the domain sequence are composed of only alanine residues, x/y is preferably 46.4% or more, more preferably 50% or more, still more preferably 55% or more, even still more preferably 60% or more, still further preferably 70% or more, and particularly preferably 80% or more. The upper limit of x/y is not particularly limited as long as it is 100% or less.

The third modified fibroin, for example, can be obtained by deleting one or a plurality sequences encoding (A)ₙ motif from a cloned gene sequence of naturally occurring fibroin such that x/y is 64.2% or more. Alternatively, the modified fibroin having a reduced content of the (A)ₙ motif may also be obtained, for example, by designing an amino acid sequence equivalent to an amino acid sequence obtained by deleting one or a plurality (A)ₙ motifs so that x/y is 64.2% or more based on the amino acid sequence of a naturally occurring fibroin and chemically synthesizing a nucleic acid encoding the designed amino acid sequence. In any case, with respect to the amino acid sequence of a naturally occurring fibroin, in addition to the modification corresponding to the deletion of the (A)ₙ motif, further modification of amino acid sequence equivalent to substitution, deletion, insertion and/or addition of one or a plurality of amino acid residues may be carried out.

A more specific example of the third modified fibroin may be a modified fibroin including (3-i) the amino acid sequence set forth in SEQ ID NO: 18, SEQ ID NO: 7, SEQ ID NO: 8, or SEQ ID NO: 9, or (3-ii) an amino acid sequence having 90% or more sequence identity with the amino acid sequence set forth in SEQ ID NO: 18, SEQ ID NO: 7, SEQ ID NO: 8, or SEQ ID NO: 9.

The modified fibroin of (3-i) will be described. The amino acid sequence set forth in SEQ ID NO: 18 is obtained by deleting one of every two (A)ₙ motifs from the N-terminal side to the C-terminal side in the amino acid sequence set forth in SEQ ID NO: 10 equivalent to a naturally occurring fibroin and by further inserting one [(A)ₙ motif−REP] just before the C-terminal sequence. The amino acid sequence set forth in SEQ ID NO: 7 is obtained by substituting all GGXs in REP of the amino acid sequence set forth in SEQ ID NO: 18 with GQX. The amino acid sequence set forth in SEQ ID NO: 8 is obtained by inserting two alanine residues at the C-terminal side of each (A)ₙ motif of the amino acid sequence set forth in SEQ ID NO: 7, and further substituting a part of glutamine (Q) residues with serine (S) residues and deleting a part of amino acids on the N-terminal side so that the molecular weight thereof is approximately the same as that of SEQ ID NO: 7. The amino acid sequence set forth in SEQ ID NO: 9 is an amino acid sequence obtained by adding a His tag to the C-terminal of a sequence obtained by repeating, four times, a region of 20 domain sequences (where several amino acid residues on the C-terminal side of the region are substituted) present in the amino acid sequence set forth in SEQ ID NO: 11.

The value of x/y with a Giza ratio of 1:1.8 to 11.3 in the amino acid sequence set forth in SEQ ID NO: 10 (equivalent to a naturally occurring fibroin) is 15.0%. Both the values of x/y in the amino acid sequences set forth in SEQ ID NO: 18 and the value of x/y in the amino acid sequence set forth in SEQ ID NO: 7 are 93.4%. The value of x/y in the amino acid sequence set forth in SEQ ID NO: 8 is 92.7%. The value of x/y in the amino acid sequence set forth in SEQ ID NO: 9 is 89.3%. The values of z/w in the amino acid sequences set forth in SEQ ID NO: 10, SEQ ID NO: 18, SEQ ID NO: 7, SEQ ID NO: 8, and SEQ ID NO: 9 are respectively 46.8%, 56.2%, 70.1%, 66.1%, and 70.0%.

The modified fibroin of (3-i) may consist of the amino acid sequence set forth in SEQ ID NO: 18, SEQ ID NO: 7, SEQ ID NO: 8, or SEQ ID NO: 9.

The modified fibroin of (3-ii) includes an amino acid sequence having 90% or more sequence identity with the amino acid sequence set forth in SEQ ID NO: 18, SEQ ID NO: 7, SEQ ID NO: 8, or SEQ ID NO: 9. The modified fibroin of (3-ii) is also a protein including a domain sequence represented by Formula 1: [(A)ₙ motif−REP]ₘ. The sequence identity is preferably 95% or more.

The modified fibroin of (3-ii) preferably has 90% or more sequence identity with the amino acid sequence set forth in SEQ ID NO: 18, SEQ ID NO: 7, SEQ ID NO: 8, or SEQ ID NO: 9, and in a case where the number of amino acid residues of two [(A)ₙ motif−REP] units adjacent to each other is sequentially compared from the N-terminal side to the C-terminal side, then the number of amino acid residues of one REP having a small number of amino acid residues is set to 1, and the maximum total value of the added numbers of amino acid residues of two [(A)ₙ motif−REP] units adjacent to each other, in which the ratio (1:1.8 to 11.3 as a Giza ratio) of the number of amino acid residues of the other REP is 1.8 to 11.3, is denoted by x, and the total number of amino acid residues in the domain sequence is denoted by y, x/y is preferably 64.2% or more.

The third modified fibroin may include a tag sequence described above at either or both of the N-terminal and C-terminal.

A more specific example of the third modified fibroin including a tag sequence may be a modified fibroin including (3-iii) the amino acid sequence set forth in SEQ ID NO: 17, SEQ ID NO: 11, SEQ ID NO: 14, or SEQ ID NO: 15, or (3-iv) an amino acid sequence having 90% or more sequence identity with the amino acid sequence set forth in SEQ ID NO: 17, SEQ ID NO: 11, SEQ ID NO: 14, or SEQ ID NO: 15.

The amino acid sequences set forth in SEQ ID NO: 16, SEQ ID NO: 17, SEQ ID NO: 13, SEQ ID NO:11, SEQ ID NO: 14, and SEQ ID NO: 15 are respectively amino acid sequences obtained by adding the amino acid sequence (including a His tag and a hinge sequence) set forth in SEQ ID NO: 12 to the N-terminal of the amino acid sequences set forth in SEQ ID NO: 10, SEQ ID NO: 18, SEQ ID NO: 6, SEQ ID NO: 7, SEQ ID NO: 8, and SEQ ID NO: 9.

The modified fibroin of (3-iii) may consist of the amino acid sequence set forth in SEQ ID NO: 17, SEQ ID NO: 11, SEQ ID NO: 14, or SEQ ID NO: 15.

The modified fibroin of (3-iv) includes an amino acid sequence having 90% or more sequence identity with the amino acid sequence set forth in SEQ ID NO: 17, SEQ ID NO: 11, SEQ ID NO: 14, or SEQ ID NO: 15. The modified fibroin of (3-iv) is also a protein including a domain sequence represented by Formula 1: [(A)ₙ motif−REP]ₘ. The sequence identity is preferably 95% or more.

The modified fibroin of (3-iv) preferably has 90% or more sequence identity with the amino acid sequence set forth in SEQ ID NO: 17, SEQ ID NO: 11, SEQ ID NO: 14, or SEQ ID NO: 15, and in a case where the number of amino acid residues of two [(A)ₙ motif−REP] units adjacent to each other is sequentially compared from the N-terminal side to the C-terminal side, then the number of amino acid residues of one REP having a small number of amino acid residues is set to 1, the maximum total value of the added numbers of amino acid residues of two [(A)ₙ motif−REP] units adjacent to each other, in which the ratio of the number of amino acid residues of the other REP is 1.8 to 11.3, is denoted by x, and the total number of amino acid residues in the domain sequence is denoted by y, x/y is preferably 64.2% or more.

The third modified fibroin may include a secretory signal for releasing the protein produced in the recombinant protein production system to the outside of a host. The sequence of the secretory signal can be appropriately set depending on the type of the host.

A domain sequence of a modified fibroin (fourth modified fibroin) having a reduced content of the glycine residue and (A)ₙ motif has an amino acid sequence having not only a reduced content of the (A)ₙ motif but also having a reduced content of the glycine residue, as compared with a naturally occurring fibroin. It can be said that the fourth modified fibroin has an amino acid sequence equivalent to an amino acid sequence in which at least one or a plurality of (A)ₙ motifs are deleted and at least one or a plurality of glycine residues in REP are further substituted with other amino acid residues, as compared with naturally occurring fibroin. That is, the fourth modified fibroin is a modified fibroin having both of the above-described characteristics of the modified fibroin (the second modified fibroin) having a reduced content of the glycine residue and the characteristics of the modified fibroin (the third modified fibroin) having a reduced content of the (A)ₙ motif. Specific aspects and the like of the fourth modified fibroin are as described in the second modified fibroin and the third modified fibroin.

A more specific example of the fourth modified fibroin includes a modified fibroin including (4-i) the amino acid sequence set forth in SEQ ID NO: 7, SEQ ID NO: 8, or SEQ ID NO: 9, or (4-ii) an amino acid sequence having 90% or more sequence identity with the amino acid sequence set forth in SEQ ID NO: 7, SEQ ID NO: 8, or SEQ ID NO: 9. Specific aspects of the modified fibroin including the amino acid sequence set forth in SEQ ID NO: 7, SEQ ID NO: 8, or SEQ ID NO: 9 are as described above.

In a modified fibroin (fifth modified fibroin) having a domain sequence including a region locally having a high hydropathy index, the domain sequence of the modified fibroin may have an amino acid sequence including the region locally having a high hydropathy index, the amino acid sequence being equivalent to an amino acid sequence in which one or a plurality of amino acid residues in REP are substituted with amino acid residues with a high hydropathy index and/or one or a plurality of amino acid residues with a high hydropathy index are inserted into REP, as compared with naturally occurring fibroin.

It is preferable that the region locally having high hydropathy index is composed of two to four consecutive amino acid residues.

It is more preferable that the above-described amino acid residues with a high hydropathy index are selected from isoleucine (I), valine (V), leucine (L), phenylalanine (F), cysteine (C), methionine (M), and alanine (A).

The fifth modified fibroin may further include an amino acid sequence equivalent to an amino acid sequence in which one or a plurality of amino acid residues are substituted, deleted, inserted and/or added, as compared with naturally occurring fibroin, in addition to the amino acid sequence in which one or a plurality of amino acid residues in REP are substituted with amino acid residues with a high hydropathy index and/or one or a plurality of amino acid residues with a high hydropathy index are inserted into REP, as compared with naturally occurring fibroin.

The fifth modified fibroin may be obtained by, with respect to a cloned gene sequence of naturally occurring fibroin, substituting one or a plurality of hydrophilic amino acid residues in REP (for example, amino acid residues having a negative hydropathy index) with a hydrophobic amino acid residue (for example, amino acid residues having a positive hydropathy index), and/or inserting one or a plurality of hydrophobic amino acid residues into REP. Further, for example, the modified fibroin may also be obtained by designing an amino acid sequence equivalent to an amino acid sequence in which with respect to the amino acid sequence of a naturally occurring fibroin, one or a plurality of hydrophilic amino acid residues in REP are substituted with hydrophobic amino acid residues and/or one or a plurality of hydrophobic amino acid residues are inserted into REP, and chemically synthesizing a nucleic acid encoding the designed amino acid sequence. In any case, with respect to the amino acid sequence a naturally occurring fibroin, in addition to the modification corresponding to the substitution of one or a plurality of hydrophilic amino acid residues in REP with hydrophobic amino acid residues and/or insertion of one or a plurality of hydrophobic amino acid residues into REP, further modification of amino acid sequence equivalent to substitution, deletion, insertion and/or addition of one or a plurality of amino acid residues may be carried out.

A fifth modified fibroin may include a domain sequence represented by Formula 1: [(A)ₙ motif−REP]ₘ and have an amino acid sequence in which p/q is 6.2% or more, in a case where in all REPs included in a sequence excluding a sequence from an (A)ₙ motif located to most C-terminal side to the C-terminal of the domain sequence from the domain sequence, the total number of amino acid residues contained in a region where an average value of hydropathy indices of four consecutive amino acid residues is 2.6 or more is denoted by p, and the total number of amino acid residues contained in the sequence excluding the sequence from the (A)ₙ motif located the most C-terminal side to the C-terminal of the domain sequence from the domain sequence is denoted by q.

Regarding the hydropathy index of amino acid residues, known indices from (Hydropathy index: Kyte J, & Doolittle R (1982)“A simple method for displaying the hydropathic character of a protein”, J. Mol. Biol., 157, pp. 105-132) may be used as a reference. Specifically, the hydropathy index (hereinafter, also referred to as “HI”) of each amino acid is as shown in Table 1 below.

TABLE 1

| Amino acid | HI |
|---|---|
| Isoleucine (Ile) | 4.5 |
| Valine (Val) | 4.2 |
| Leucine (Leu) | 3.8 |
| Phenylalanine (Phe) | 2.8 |
| Cysteine (Cys) | 2.5 |
| Methionine (Met) | 1.9 |
| Alanine (Ala) | 1.8 |
| Glycine (Gly) | −0.4 |
| Threonine (Thr) | −0.7 |
| Serine (Ser) | −0.8 |
| Tryptophan (Trp) | −0.9 |
| Tyrosine (Tyr) | −1.3 |
| Proline (Pro) | −1.6 |
| Histidine (His) | −3.2 |
| Asparagine (Asn) | −3.5 |
| Aspartic acid (Asp) | −3.5 |
| Glutamine (Gln) | −3.5 |
| Glutamic acid (Glu) | −3.5 |
| Lysine (Lys) | −3.9 |
| Arginine (Arg) | −4.5 |

The calculation method for p/q will be described in more detail. In the calculation, the sequence (hereinafter, also referred to as “sequence A”) excluding a sequence from the (A)ₙ motif located closest to the C-terminal side to the C-terminal of the domain sequence from the domain sequence represented by Formula 1: ([(A)ₙ motif−REP]ₘ−(A)ₙ motif] is used. First, in all REPs included in the sequence A, average values of hydropathy indices of the four consecutive amino acid residues are calculated. The average value of the hydropathy indices is obtained by dividing the total sum of HI of each of the amino acid residues contained in the four consecutive amino acid residues by 4 (the number of amino acid residues). The average value of the hydropathy indices is obtained for all of the four consecutive amino acid residues (each of the amino acid residues is used for calculating the average value 1 to 4 times). Next, a region where the average value of the hydropathy indices of the four consecutive amino acid residues is 2.6 or more is specified. Even in a case where a plurality of certain amino acid residues correspond to the “four consecutive amino acid residues having an average value of the hydropathy indices of 2.6 or more”, the amino acid residue is counted as one amino acid residue in the region. The total number of amino acid residues included in the region is denoted by p. The total number of amino acid residues included in the sequence A is denoted by q.

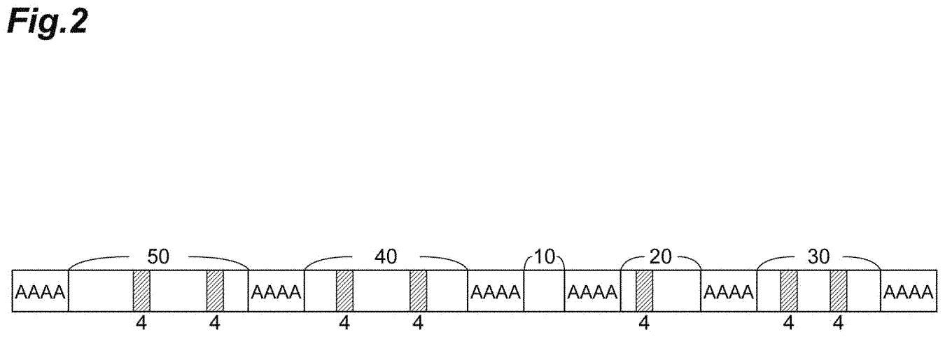

For example, in a case where the “four consecutive amino acid residues whose average value of the hydropathy indices is 2.6 or more” are extracted from 20 places (without overlap), in the region where the average value of the hydropathy indices of the four consecutive amino acid residues is 2.6 or more, the number of the four consecutive amino acid residues (without overlap) is 20, and thus p is 20×4=80. In addition, for example, in a case where two of the “four consecutive amino acid residues having an average value of the hydropathy indices of 2.6 or more” overlap by only one amino acid residue, in the region where the average value of the hydropathy indices of the four consecutive amino acid residues is 2.6 or more, the number of amino acid residues being included is 7 (p=2×4−1=7. “−1” corresponds to the subtraction of the overlapping portion). For example, in the case of the domain sequence shown in FIG. 2, since the number of the “four consecutive amino acid residues having an average value of the hydropathy indices of 2.6 or more”, which do not overlap, is 7, p is 7×4=28. Further, for example, in the case of the domain sequence illustrated in FIG. 2, q is 4+50+4+40+4+10+4+20+4+30=170 (the (A)ₙ motif present closest to the C-terminal side cannot be included). Next, p/q (%) can be calculated by dividing p by q. In the case of FIG. 2, p/q (%) is 28/170=16.47%.

In the fifth modified fibroin, p/q is preferably 6.2% or more, more preferably 7% or more, still more preferably 10% or more, even still more preferably 20% or more, and still further preferably 30% or more. The upper limit of p/q is not particularly limited, but for example, it may be 45% or less.

The fifth modified fibroin may be obtained by, for example, modifying an amino acid sequence of cloned naturally occurring fibroin into an amino acid sequence locally containing a region having a high hydropathy index by substituting one or a plurality of hydrophilic amino acid residues in REP (for example, amino acid residues having a negative hydropathy index) with hydrophobic amino acid residues (for example, amino acid residues having a positive hydropathy index), and/or inserting one or a plurality of hydrophobic amino acid residues into REP, such that the p/q condition is satisfied. Alternatively, the modified fibroin may also be obtained, for example, by designing an amino acid sequence satisfying the p/q condition based on the amino acid sequence of a naturally occurring fibroin and chemically synthesizing a nucleic acid encoding the designed amino acid sequence. In any case, in addition to the modification corresponding to the substitution of one or a plurality of amino acid residues in REP with amino acid residues with a high hydropathy index and/or insertion of one or a plurality of amino acid residues with a high hydropathy index into REP, as compared with the amino acid sequence of a naturally occurring fibroin, further modification corresponding to substitution, deletion, insertion, and/or addition of one or a plurality of amino acid residues may be carried out.

The amino acid residue with a high hydropathy index is preferably isoleucine (I), valine (V), leucine (L), phenylalanine (F), cysteine (C), methionine (M), and alanine (A), and more preferably valine (V), leucine (L), and isoleucine (I), but is not particularly limited thereto.

A specific example of the fourth modified fibroin includes a modified fibroin including (5-i) the amino acid sequence set forth in SEQ ID NO: 19, SEQ ID NO: 20, or SEQ ID NO: 21, or (5-ii) an amino acid sequence having 90% or more sequence identity with the amino acid sequence set forth in SEQ ID NO: 19, SEQ ID NO: 20, or SEQ ID NO: 21.

The modified fibroin of (5-i) will be described. The amino acid sequence set forth in SEQ ID NO: 22 is obtained by deleting a part of the amino acid sequence of the consecutive alanine residues in the (A)ₙ motif of a naturally occurring fibroin so that the number of the consecutive alanine residues in the (A)ₙ motif is five. The amino acid sequence set forth in SEQ ID NO: 19 is obtained by inserting an amino acid sequence consisting of three amino acid residues (VLI) at two sites for every other REP with respect to the amino acid sequence set forth in SEQ ID NO: 22, and deleting a part of the amino acids on the C-terminal side therefrom so that the molecular weight thereof is approximately the same as that of the amino acid sequence set forth in SEQ ID NO: 22. The amino acid sequence set forth in SEQ ID NO: 23 is obtained by inserting two alanine residues at the C-terminal side of each (A)ₙ motif with respect to the amino acid sequence set forth in SEQ ID NO: 22, and further substituting a part of glutamine (Q) residues with serine (S) residues and deleting a part of amino acids on the C-terminal side so that the molecular weight thereof is approximately the same as that of the amino acid sequence set forth in SEQ ID NO: 22. The amino acid sequence set forth in SEQ ID NO: 20 is obtained by inserting an amino acid sequence consisting of three amino acid residues (VLI) at one site for every other REP with respect to the amino acid sequence set forth in SEQ ID NO: 23. The amino acid sequence set forth in SEQ ID NO: 21 is obtained by inserting an amino acid sequence consisting of three amino acid residues (VLI) at two sites for every other REP with respect to the amino acid sequence set forth in SEQ ID NO: 23.

The modified fibroin of (5-i) may consist of the amino acid sequence set forth in SEQ ID NO: 19, SEQ ID NO: 20, or SEQ ID NO: 21.

The modified fibroin of (5-ii) includes an amino acid sequence having 90% or more sequence identity with the amino acid sequence set forth in SEQ ID NO: 19, SEQ ID NO: 20, or SEQ ID NO: 21. The modified fibroin of (5-ii) is also a protein including a domain sequence represented by Formula 1: [(A)ₙ motif−REP]ₘ. The sequence identity is preferably 95% or more.

The modified fibroin of (5-ii) preferably has 90% or more sequence identity with the amino acid sequence set forth in SEQ ID NO: 19, SEQ ID NO: 20, or SEQ ID NO: 21, and preferably has an amino acid sequence in which p/q is 6.2% or more, in a case where in all REPs included in a sequence excluding a sequence from the (A)ₙ motif located closest to the C-terminal side to the C-terminal of the domain sequence from the domain sequence, the total number of amino acid residues contained in a region where an average value of hydropathy indices of the four consecutive amino acid residues is 2.6 or more is denoted by p, and the total number of amino acid residues contained in the sequence excluding a sequence from the (A)ₙ motif located closest to the C-terminal side to the C-terminal of the domain sequence from the domain sequence is denoted by q.

The fifth modified fibroin may include a tag sequence at either or both of the N-terminal and C-terminal.

A more specific example of the fifth modified fibroin including a tag sequence may be a modified fibroin including (5-iii) the amino acid sequence set forth in SEQ ID NO: 24, SEQ ID NO: 25, or SEQ ID NO: 26, or (5-iv) an amino acid sequence having 90% or more sequence identity with the amino acid sequence set forth in SEQ ID NO: 24, SEQ ID NO: 25, or SEQ ID NO: 26.

The amino acid sequences set forth in SEQ ID NO: 24, SEQ ID NO: 25, and SEQ ID NO: 26 are respectively amino acid sequences obtained by adding the amino acid sequence (including a His tag and a hinge sequence) set forth in SEQ ID NO: 12 to the N-terminal of the amino acid sequences set forth in SEQ ID NO: 19, SEQ ID NO: 20, and SEQ ID NO: 21.

The modified fibroin of (5-iii) may consist of the amino acid sequence set forth in SEQ ID NO: 24, SEQ ID NO: 25, or SEQ ID NO: 26.

The modified fibroin of (5-iv) includes an amino acid sequence having 90% or more sequence identity with the amino acid sequence set forth in SEQ ID NO: 24, SEQ ID NO: 25, or SEQ ID NO: 26. The modified fibroin of (5-iv) is also a protein including a domain sequence represented by Formula 1: [(A)ₙ motif−REP]ₘ. The sequence identity is preferably 95% or more.

The modified fibroin of (5-iv) preferably has 90% or more sequence identity with the amino acid sequence set forth in SEQ ID NO: 24, SEQ ID NO: 25, or SEQ ID NO: 26, and preferably has an amino acid sequence in which p/q is 6.2% or more, in a case where in all REPs included in a sequence excluding a sequence from the (A)ₙ motif located closest to the C-terminal side to the C-terminal of the domain sequence from the domain sequence, the total number of amino acid residues contained in a region where an average value of hydropathy indices of the four consecutive amino acid residues is 2.6 or more is denoted by p, and the total number of amino acid residues contained in the sequence excluding a sequence from the (A)ₙ motif located closest to the C-terminal side to the C-terminal of the domain sequence from the domain sequence is denoted by q.

The fifth modified fibroin may include a secretory signal for releasing the protein produced in the recombinant protein production system to the outside of a host. The sequence of the secretory signal can be appropriately set depending on the type of the host.

A domain sequence of a modified fibroin (sixth modified fibroin) having a domain sequence with a reduced content of the glutamine residue has an amino acid sequence with a reduced content of the glutamine residue, as compared with a naturally occurring fibroin.

The sixth modified fibroin preferably includes at least one motif selected from GGX motif and GPGXX motif in the amino acid sequence of REP.

In a case where the sixth modified fibroin includes a GPGXX motif in REP, a GPGXX motif content rate is usually 1% or more, may be 5% or more, and is preferably 10% or more. The upper limit of the GPGXX motif content rate is not particularly limited, may be 50% or less, and may be 30% or less.

In the present specification, the “GPGXX motif content rate” is a value calculated by the following method. In a modified fibroin including a domain sequence represented by Formula 1: [(A)ₙ motif−REP]ₘ or Formula 2: [(A)ₙ motif−REP]ₘ−(A)ₙ motif, in a case where the number obtained by tripling the total number of the GPGXX motifs included in all REPs included in a sequence excluding the sequence from the (A)ₙ motif located at the most C-terminal side to the C-terminal of the domain sequence from the domain sequence (that is, equivalent to the total number of G and P in the GPGXX motifs) is denoted by s, and the total number of amino acid residues in all REPs excluding the sequence from the (A)ₙ motif located at the most the C-terminal side to the C-terminal of the domain sequence from the domain sequence and further excluding (A)ₙ motifs is denoted by t, the GPGXX motif content rate is calculated as s/t.

For the calculation of the GPGXX motif content rate, the “sequence excluding a sequence from the (A)ₙ motif located closest to the C-terminal side to the C-terminal of the domain sequence from the domain sequence” is used to exclude the effect occurring due to the fact that the “sequence from the (A)ₙ motif located closest to the C-terminal side to the C-terminal from the domain sequence” (sequence equivalent to REP) may include a sequence that is not correlated with the sequence characteristics of fibroin, which influences the calculation result of the GPGXX motif content rate in a case where m is small (that is, in case a where the domain sequence is short). In a case where a “GPGXX motif” is located at the C-terminal of REP, it is treated as “GPGXX motif” even in a case where “XX” is, for example, “AA”.

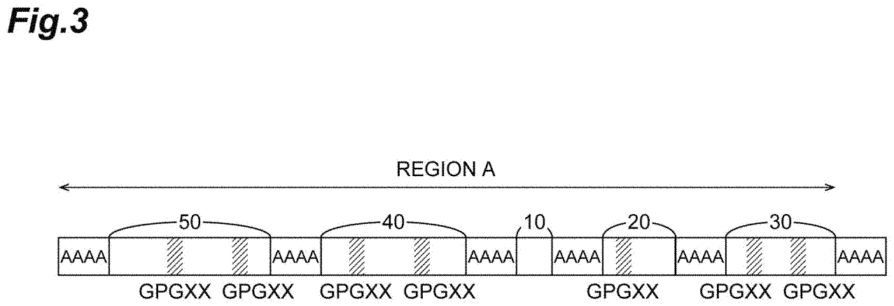

FIG. 3 is a schematic diagram showing a domain sequence of fibroin. The calculation method for the GPGXX motif content rate will be specifically described with reference to FIG. 3. First, in a domain sequence of a fibroin (which is an [(A)ₙ motif−REP]ₘ−(A)ₙ motif] type) illustrated in FIG. 3, since all REPs are included in the “sequence excluding a sequence from the (A)ₙ motif located closest to the C-terminal side to the C-terminal of the domain sequence from the domain sequence” (in FIG. 3, shown as “region A”), the number of GPGXX motifs for calculating s is 7, and s is 7×3=21. Similarly, since all REPs are included in the “sequence excluding a sequence from the (A)ₙ motif located closest to the C-terminal side to the C-terminal of the domain sequence from the domain sequence” (in FIG. 3, shown as “region A”), t which is the total number of amino acid residues in all REPs excluding a sequence from the (A)ₙ motif located closest to the C-terminal side to the C-terminal of the domain sequence from the domain sequence and further excluding (A)ₙ motifs, is 50+40+10+20+30=150. Next, s/t (%) can be calculated by dividing s by t and is 21/150=14.0% in the case of the fibroin of FIG. 3.

In the sixth modified fibroin, a glutamine residue content rate is preferably 9% or less, more preferably 7% or less, still more preferably 4% or less, and particularly preferably 0%.

In the present specification, the “glutamine residue content rate” is a value calculated by the following method. In a modified fibroin including a domain sequence represented by Formula 1: [(A)ₙ motif−REP]ₘ or Formula 2: [(A)ₙ motif−REP]ₘ−(A)ₙ motif, in a case where the total number of glutamine residues included in all REPs included in a sequence (sequence equivalent to “region A” in FIG. 3) excluding the sequence from the (A)ₙ motif located at the most C-terminal side to the C-terminal of the domain sequence from the domain sequence is denoted by u, and the total number of amino acid residues in all REPs excluding the sequence from the (A)ₙ motif located at the most the C-terminal side to the C-terminal of the domain sequence from the domain sequence and further excluding (A)ₙ motifs is denoted by t, the glutamine residue content rate is calculated as u/t. For the calculation of the glutamine residue content rate, the “sequence excluding a sequence from the (A)ₙ motif located closest to the C-terminal side to the C-terminal of the domain sequence from the domain sequence” is used for the same reason described above.

The domain sequence of the sixth modified fibroin may include an amino acid sequence equivalent to an amino acid sequence in which one or a plurality of glutamine residues in REP are deleted or substituted with other amino acid residues, as compared with a naturally occurring fibroin.

The “other amino acid residue” may be an amino acid residue other than a glutamine residue but is preferably an amino acid residue having a higher hydropathy index than that of a glutamine residue. The hydropathy indices of amino acid residues are as shown in Table 1.

As shown in Table 1, amino acid residues having a higher hydropathy index than a glutamine residue include an amino acid residue selected from isoleucine (I), valine (V), leucine (L), phenylalanine (F), cysteine (C), methionine (M), alanine (A), glycine (G), threonine (T), serine (S), tryptophan (W), tyrosine (Y), proline (P) and histidine (H). Among these, an amino acid residue selected from isoleucine (I), valine (V), leucine (L), phenylalanine (F), cysteine (C), methionine (M), and alanine (A) is more preferable, and an amino acid residue selected from isoleucine (I), valine (V), leucine (L), and phenylalanine (F) is still more preferable.

In the sixth modified fibroin, the hydrophobicity of REP is preferably −0.8 or more, more preferably −0.7 or more, still more preferably 0 or more, even still more preferably 0.3 or more, and particularly preferably 0.4 or more. The upper limit of the hydrophobicity of REP is not particularly limited, may be 1.0 or less, and may be 0.7 or less.

In the present specification, the “hydrophobicity of REP” is a value calculated by the following method. In a modified fibroin including a domain sequence represented by Formula 1: [(A)ₙ motif−REP]ₘ or Formula 2: [(A)ₙ motif−REP]ₘ−(A)ₙ motif, in a case where the sum of the hydropathy indices of each amino acid residue included in all REPs included in a sequence (sequence equivalent to “region A” in FIG. 3) excluding the sequence from the (A)ₙ motif located at the most C-terminal side to the C-terminal of the domain sequence from the domain sequence is denoted by v, and the total number of amino acid residues in all REPs excluding the sequence from the (A)ₙ motif located at the most the C-terminal side to the C-terminal of the domain sequence from the domain sequence and further excluding (A)ₙ motifs is denoted by t, the hydrophobicity of REP is calculated as v/t. For the calculation of the hydrophobicity of REP, the “sequence excluding a sequence from the (A)ₙ motif located closest to the C-terminal side to the C-terminal of the domain sequence from the domain sequence” is used for the same reason described above.

The domain sequence of the sixth modified fibroin may further include an amino acid sequence equivalent to an amino acid sequence in which one or a plurality of amino acid residues are substituted, deleted, inserted and/or added, in addition to the modification of the amino acid sequence in which one or a plurality of glutamine residues in REP are deleted and/or one or a plurality of glutamine residues in REP are substituted with other amino acid residues, as compared with a naturally occurring fibroin.

The sixth modified fibroin can be obtained by, for example, with respect to a cloned gene sequence of a naturally occurring fibroin, deleting one or a plurality of glutamine residues in REP and/or by substituting one or a plurality of glutamine residues in REP with other amino acid residues. Further, for example, the modified fibroin may also be obtained by designing an amino acid sequence equivalent to an amino acid sequence in which with respect to the amino acid sequence of a naturally occurring fibroin, one or a plurality of glutamine residues in REP are deleted and/or one or a plurality of glutamine residues in REP are substituted with other amino acid residues, and chemically synthesizing a nucleic acid encoding the designed amino acid sequence.

A more specific example of the sixth modified fibroin may be a modified fibroin including (6-i) the amino acid sequence set forth in SEQ ID NO: 27, SEQ ID NO: 28, SEQ ID NO: 29, SEQ ID NO: 30, SEQ ID NO: 31, SEQ ID NO: 32, or SEQ ID NO: 33, or (6-ii) an amino acid sequence having 90% or more sequence identity with the amino acid sequence set forth in SEQ ID NO: 27, SEQ ID NO: 28, SEQ ID NO: 29, SEQ ID NO: 30, SEQ ID NO: 31, SEQ ID NO: 32, or SEQ ID NO: 33.

The modified fibroin of (6-i) will be described.

The amino acid sequence (Met-PRT410) set forth in SEQ ID NO: 7 is a modified amino acid sequence obtained by changing the number of the consecutive alanine residues in the (A)ₙ motif to five, or the like, so as to improve productivity, based on the base sequence and amino acid sequence of Nephila clavipes (GenBank Accession No.: P46804.1, GI: 1174415) which is a naturally occurring fibroin. However, since Met-PRT410 has no modification of glutamine residue (Q), the glutamine residue content rate thereof is the same as the glutamine residue content of a naturally occurring fibroin.

The amino acid sequence (M_PRT888) set forth in SEQ ID NO: 27 is obtained by substituting all QQs in Met-PRT410 (SEQ ID NO: 7) with VLs.

The amino acid sequence (M_PRT965) set forth in SEQ ID NO: 28 is obtained by substituting all QQs in Met-PRT410 (SEQ ID NO: 7) with TSs and substituting the remaining Qs with As.

The amino acid sequence (M_PRT889) set forth in SEQ ID NO: 29 is obtained by substituting all QQs in Met-PRT410 (SEQ ID NO: 7) with VLs and substituting the remaining Qs with Is.

The amino acid sequence (M_PRT916) set forth in SEQ ID NO: 30 is obtained by substituting all QQs in Met-PRT410 (SEQ ID NO: 7) with VIs and substituting the remaining Qs with Ls.

The amino acid sequence (M_PRT918) set forth in SEQ ID NO: 31 is obtained by substituting all QQs in Met-PRT410 (SEQ ID NO: 7) with VFs and substituting the remaining Qs with Is.

The amino acid sequence (M_PRT525) set forth in SEQ ID NO: 34 is obtained by, with respect to Met-PRT410 (SEQ ID NO: 7), inserting two alanine residues in a region (A5) in which alanine residues are consecutive, and by deleting two domain sequences at the C-terminal side and substituting 13 glutamine residues (Q) with serine residues (S) or prolines (P) so that the molecular weight thereof is approximately the same as that of Met-PRT410.

The amino acid sequence (M_PRT699) set forth in SEQ ID NO: 32 is obtained by substituting all QQs in M_PRT525 (SEQ ID NO: 34) with VLs.

The amino acid sequence (M_PRT698) set forth in SEQ ID NO: 33 is obtained by substituting all QQs in M_PRT525 (SEQ ID NO: 34) with VLs and substituting the remaining Qs with Is.

The glutamine residue content rate of any of the amino acid sequences set forth in SEQ ID NO: 27, SEQ ID NO: 28, SEQ ID NO: 29, SEQ ID NO: 30, SEQ ID NO: 31, SEQ ID NO: 32, and SEQ ID NO: 33 is 9% or less (Table 2).

TABLE 2

| | Glutamine | | |
|---|---|---|---|
| | residue | GPGXX motif | Hydrophobicity |
| Modified fibroin | content rate | content rate | of REP |
| Met-PRT410 (SEQ ID | 17.7% | 27.9% | −1.52 |
| NO: 7) | | | |
| M_PRT888 (SEQ ID | 6.3% | 27.9% | −0.07 |
| NO: 27) | | | |
| M_PRT965 (SEQ ID | 0.0% | 27.9% | −0.65 |
| NO: 28) | | | |
| M_PRT889 (SEQ ID | 0.0% | 27.9% | 0.35 |
| NO: 29) | | | |
| M_PRT916 (SEQ ID | 0.0% | 27.9% | 0.47 |
| NO: 30) | | | |
| M_PRT918 (SEQ ID | 0.0% | 27.9% | 0.45 |
| NO: 31) | | | |
| M_PRT525 (SEQ ID | 13.7% | 26.4% | −1.24 |
| NO: 34) | | | |
| M_PRT699 (SEQ ID | 3.6% | 26.4% | −0.78 |
| NO: 32) | | | |
| M_PRT698 (SEQ ID | 0.0% | 26.4% | −0.03 |
| NO: 33) | | | |

The modified fibroin of (6-i) may consist of the amino acid sequence set forth in SEQ ID NO: 27, SEQ ID NO: 28, SEQ ID NO: 29, SEQ ID NO: 30, SEQ ID NO: 31, SEQ ID NO: 32, or SEQ ID NO: 33.

The modified fibroin of (6-ii) includes an amino acid sequence having 90% or more sequence identity with the amino acid sequence set forth in SEQ ID NO: 27, SEQ ID NO: 28, SEQ ID NO: 29, SEQ ID NO: 30, SEQ ID NO: 31, SEQ ID NO: 32, or SEQ ID NO: 33. The modified fibroin of (6-ii) is also a protein including a domain sequence represented by Formula 1: [(A)ₙ motif−REP]ₘ or Formula 2: [(A)ₙ motif−REP]ₘ−(A)ₙ motif. The sequence identity is preferably 95% or more.

The modified fibroin of (6-ii) preferably has the glutamine residue content rate of 9% or less. In addition, the modified fibroin of (6-ii) preferably has the GPGXX motif content rate of 10% or more.

The sixth modified fibroin may include a tag sequence at either or both of the N-terminal and C-terminal. This makes it possible to isolate, immobilize, detect, and visualize the modified fibroin.

A more specific example of the sixth modified fibroin including a tag sequence includes a modified fibroin including (6-iii) the amino acid sequence set forth in SEQ ID NO: 35, SEQ ID NO: 36, SEQ ID NO: 37, SEQ ID NO: 38, SEQ ID NO: 39, SEQ ID NO: 40, or SEQ ID NO: 41, or (6-iv) an amino acid sequence having 90% or more sequence identity with the amino acid sequence set forth in SEQ ID NO: 35, SEQ ID NO: 36, SEQ ID NO: 37, SEQ ID NO: 38, SEQ ID NO: 39, SEQ ID NO: 40, or SEQ ID NO: 41.

The amino acid sequences set forth in SEQ ID NO: 35, SEQ ID NO: 36, SEQ ID NO: 37, SEQ ID NO: 38, SEQ ID NO: 39, SEQ ID NO: 40, and SEQ ID NO: 41 are respectively amino acid sequences obtained by adding the amino acid sequence (including a His tag and a hinge sequence) set forth in SEQ ID NO: 12 to the N-terminal of the amino acid sequences set forth in SEQ ID NO: 27, SEQ ID NO: 28, SEQ ID NO: 29, SEQ ID NO: 30, SEQ ID NO: 31, SEQ ID NO: 32, and SEQ ID NO: 33. Since only the tag sequence is added to the N-terminal, the glutamine residue content rate are not changed, and any of the amino acid sequences set forth in SEQ ID NO: 35, SEQ ID NO: 36, SEQ ID NO: 37, SEQ ID NO: 38, SEQ ID NO: 39, SEQ ID NO: 40, and SEQ ID NO: 41 has the glutamine residue content rate of 9% or less (Table 3).

TABLE 3

| | Glutamine | | |
|---|---|---|---|
| | residue | GPGXX motif | Hydrophobicity |
| Modified fibroin | content rate | content rate | of REP |
| PRT888 (SEQ ID | 6.3% | 27.9% | −0.07 |
| NO: 35) | | | |
| PRT965 (SEQ ID | 0.0% | 27.9% | −0.65 |
| NO: 36) | | | |
| PRT889 (SEQ ID | 0.0% | 27.9% | 0.35 |
| NO: 37) | | | |
| PRT916 (SEQ ID | 0.0% | 27.9% | 0.47 |
| NO: 38) | | | |
| PRT918 (SEQ ID | 0.0% | 27.9% | 0.45 |
| NO: 39) | | | |
| PRT699 (SEQ ID | 3.6% | 26.4% | −0.78 |
| NO: 40) | | | |
| PRT698 (SEQ ID | 0.0% | 26.4% | −0.03 |
| NO: 41) | | | |

The modified fibroin of (6-iii) may consist of the amino acid sequence set forth in SEQ ID NO: 35, SEQ ID NO: 36, SEQ ID NO: 37, SEQ ID NO: 38, SEQ ID NO: 39, SEQ ID NO: 40, or SEQ ID NO: 41.

The modified fibroin of (6-iv) includes an amino acid sequence having 90% or more sequence identity with the amino acid sequence set forth in SEQ ID NO: 35, SEQ ID NO: 36, SEQ ID NO: 37, SEQ ID NO: 38, SEQ ID NO: 39, SEQ ID NO: 40, or SEQ ID NO: 41. The modified fibroin of (6-iv) is also a protein including a domain sequence represented by Formula 1: [(A)ₙ motif−REP]ₘ or Formula 2: [(A)ₙ motif−REP]ₘ−(A)ₙ motif. The sequence identity is preferably 95% or more.

The modified fibroin of (6-iv) preferably has the glutamine residue content rate of 9% or less. In addition, the modified fibroin of (6-iv) preferably has the GPGXX motif content rate of 10% or more.

The sixth modified fibroin may include a secretory signal for releasing the protein produced in the recombinant protein production system to the outside of a host. The sequence of the secretory signal can be appropriately set depending on the type of the host.

The modified fibroin according to the present embodiment may be a modified fibroin having at least two or more characteristics among the characteristics of the first modified fibroin, the second modified fibroin, the third modified fibroin, the fourth modified fibroin, the fifth modified fibroin, and the sixth modified fibroin.

Examples of proteins derived from collagen include a protein including a domain sequence represented by Formula 3: [REP2]ₚ (Here in Formula 3, p represents an integer of 5 to 300. REP2 represents an amino acid sequence composed of Gly-X-Y, and X and Y represent any amino acid residue other than Gly. A plurality of REP2s may have the same amino acid sequence or amino acid sequences different from each other.). Specifically, a protein including the amino acid sequence set forth in SEQ ID NO: 42 can be mentioned. The amino acid sequence set forth in SEQ ID NO: 42 is obtained by adding the amino acid sequence set forth in SEQ ID NO: 12 (a tag sequence and a hinge sequence) to the N-terminal of the amino acid sequence from the 301th residue to the 540th residue, which corresponds to the repeat portion and motif of the partial sequence of human collagen type 4 (NCBI GenBank Accession No.: CAA56335.1, GI: 3702452) obtained from the NCBI database.

Examples of proteins derived from resilin include a protein including a domain sequence represented by the formula 4: [REP3]_{q} (Here in Formula 4, q represents an integer of 4 to 300. REP3 represents an amino acid sequence composed of Ser-J-J-J-Tyr-Gly-U-Pro. J represents any amino acid residue and is particularly preferably an amino acid residue selected from the group consisting of Asp, Ser, and Thr. U represents any amino acid residue and is particularly an amino acid residue selected from the group consisting of Pro, Ala, Thr, and Ser. A plurality of REP4s may have the same amino acid sequence or amino acid sequences different from each other. Specifically, a protein including the amino acid sequence set forth in SEQ ID NO: 43 can be mentioned. The amino acid sequence set forth in SEQ ID NO: 43 is obtained by adding the amino acid sequence set forth in SEQ ID NO: 12 (a tag sequence and a hinge sequence) to the N-terminal of the amino acid sequence from 19th residue to 321th residue of the amino acid sequence of resilin (NCBI GenBank Accession No. NP611157, Gl: 24654243), in which Thr at the 87th residue is substituted with Ser, and Asn at the 95th residue is substituted with Asp.

Examples of proteins derived from elastin include proteins having amino acid sequences such as NCBI GenBank Accession No.s, AAC98395 (human), 147076 (sheep), and NP786966 (bovine). Specifically, a protein including the amino acid sequence set forth in SEQ ID NO: 44 can be mentioned. The amino acid sequence set forth in SEQ ID NO: 44 is obtained by adding the amino acid sequence set forth in SEQ ID NO: 12 (a tag sequence and a hinge sequence) to the N-terminal of the amino acid sequence from 121th residue to 390th residue of the amino acid sequence of NCBI GenBank Accession No. AAC98395.

Examples of proteins derived from keratin include a type I keratin of Capra hircus. Specifically, a protein including the amino acid sequence set forth in SEQ ID NO: 45 (the amino acid sequence of NCBI GenBank Accession No. ACY30466) can be mentioned.

The structural proteins and the modified structural proteins derived from the structural proteins described above can be used alone or in a combination of two or more thereof.

(Production Method for Protein)

A protein can be produced, for example, by expressing a nucleic acid in a host transformed with an expression vector having a nucleic acid sequence encoding the protein and one or a plurality of regulatory sequences operably linked to the nucleic acid sequence.

The production method for a nucleic acid encoding a protein is not particularly limited. For example, the nucleic acid is produced by cloning a gene encoding a protein such as the natural fibroin by amplification with polymerase chain reaction (PCR) or the like and, as necessary, modifying the gene by a genetic engineering method, by chemically synthesizing the nucleic acid. The method for chemically synthesizing a nucleic acid is not particularly limited, and for example, the gene can be chemically synthesized by a method in which oligonucleotides are automatically synthesized by AKTA oligopilot plus 10/100 (GE Healthcare Japan Corporation) or the like and are linked by PCR or the like, based on the amino acid sequence information of the protein obtained from the NCBI web database or the like. In this case, in order to facilitate purification and/or confirmation of the protein, a nucleic acid may be synthesized such that a protein having an amino acid sequence obtained by adding an amino acid sequence consisting of a start codon and a His10 tag to the N-terminal of the above amino acid sequence is encoded.

The regulatory sequence is a sequence (for example, a promoter, an enhancer, a ribosome binding sequence, or a transcription termination sequence) that controls the expression of a protein in a host, and can be appropriately selected depending on the type of the host. As a promoter, an inducible promoter that functions in a host cell and is capable of inducing the expression of a protein may be used. An inducible promoter is a promoter that can control transcription by the presence of an inducer (an expression inducer), the absence of a repressor molecule, or physical factors such as an increase or decrease in temperature, osmotic pressure, or pH value.

The type of the expression vector such as a plasmid vector, a viral vector, a cosmid vector, a fosmid vector, or an artificial chromosome vector can be appropriately selected depending on the type of the host. As the expression vector, an expression vector that can autonomously replicate in a host cell or can be incorporated into a chromosome of a host and which contains a promoter at a position capable of transcribing the nucleic acid that encodes a protein is suitably used.

Both prokaryotes and eukaryotes such as yeast, filamentous fungi, insect cells, animal cells, and plant cells can be suitably used as a host.

Preferred examples of the prokaryotic host cells include bacteria belonging to the genus Escherichia, the genus Brevibacillus, the genus Serratia, the genus Bacillus, the genus Microbacterium, the genus Brevibacterium, the genus Corynebacterium, and the genus Pseudomonas. Examples of microorganisms belonging to the genus Escherichia include Escherichia coli. Examples of the microorganisms belonging to the genus Brevibacillus include Brevibacillus agri. Examples of microorganisms belonging to the genus Serratia include Serratia liquefaciens. Examples of microorganisms belonging to the genus Bacillus include Bacillus subtilis. Examples of microorganisms belonging to the genus Microbacterium include Microbacterium ammoniaphilum. Examples of microorganisms belonging to the genus Brevibacterium include Brevibacterium divaricatum. Examples of microorganisms belonging to the genus Corynebacterium include Corynebacterium ammoniagenes. Examples of microorganisms belonging to the genus Pseudomonas include Pseudomonas putida.

In a case where a prokaryote is used as a host, examples of a vector into which a nucleic acid encoding a protein is introduced include pBTrp2 (manufactured by Boehringer Mannheim), pGEX (manufactured by Pharmacia), pUC18, pBluescriptII, pSupex, pET22b, pCold, pUB110, and pNCO2 (Japanese Unexamined Patent Publication No. 2002-238569).

Examples of eukaryotic hosts include yeast and filamentous fungi (mold and the like). Examples of yeasts include yeasts belonging to the genus Saccharomyces, the genus Pichia, and the genus Schizosaccharomyces. Examples of filamentous fungi include filamentous fungi belonging to the genus Aspergillus, the genus Penicillium, and the genus Trichoderma.

In a case where a eukaryote is used as a host, examples of the vector into which a nucleic acid encoding a protein is introduced include YEp13 (ATCC37115) and YEp24 (ATCC37051). As a method for introducing an expression vector into the above host cell, any method can be used as long as the method introduces DNA into the host cell. Examples thereof include a method using calcium ions [Proc. Natl. Acad. Sci. USA, 69, 2110 (1972)], electroporation method, spheroplast method, protoplast method, lithium acetate method, and competent method.

As for the method for expressing a nucleic acid using a host transformed with an expression vector, secretory production, fusion protein expression, or the like, in addition to the direct expression, can be carried out according to the method described in Molecular Cloning, 2nd edition.

The protein can be produced, for example, by culturing a host transformed with the expression vector in a culture medium, producing and accumulating the protein in the culture medium, and then collecting the modified fibroin from the culture medium. The method for culturing a host in a culture medium can be carried out according to a method commonly used for culturing a host.

In the case where the host is a prokaryote such as Escherichia coli or a eukaryote such as yeast, any of a natural medium and a synthetic medium may be used as a culture medium of the host as long as the medium contains a carbon source, a nitrogen source, inorganic salts and the like which can be utilized by the host and the medium can be used for efficiently culturing the host.

As the carbon source, any carbon source that can be utilized by the transformed microorganism may be used. Examples of the carbon source that can be utilized include glucose, fructose, sucrose, and molasses containing them, carbohydrates such as starch and a hydrolyzate thereof, organic acids such as acetic acid and propionic acid, and alcohols such as ethanol and propanol. Examples of the nitrogen source that can be utilized include ammonium salts of inorganic or organic acids such as ammonia, ammonium chloride, ammonium sulfate, ammonium acetate, and ammonium phosphate, other nitrogen-containing compounds, peptone, meat extract, yeast extract, corn steep liquor, casein hydrolyzate, soybean cake and soybean cake hydrolyzate, and various fermented microbial cells and digested products thereof. Examples of the inorganic salt that can be utilized include potassium dihydrogen phosphate, dipotassium phosphate, magnesium phosphate, magnesium sulfate, sodium chloride, ferrous sulfate, manganese sulfate, copper sulfate, and calcium carbonate.

Culture of a prokaryote such as Escherichia coli or a eukaryote such as yeast can be carried out under aerobic conditions such as shaking culture or deep aeration stirring culture. The culture temperature is, for example, 15° C. to 40° C. The culture time is usually 16 hours to 7 days. It is preferable to maintain the pH of the culture medium during the culture at 3.0 to 9.0. The pH of the culture medium can be adjusted using an inorganic acid, an organic acid, an alkali solution, urea, calcium carbonate, ammonia, or the like.

In addition, antibiotics such as ampicillin and tetracycline may be added to the culture medium as necessary during the culture. In a case of culturing a microorganism transformed with an expression vector using an inducible promoter as a promoter, an inducer may be added to the medium as necessary. For example, in a case of culturing a microorganism transformed with an expression vector using a lac promoter, isopropyl-β-D-thiogalactopyranoside or the like is used, and in a case of culturing a microorganism transformed with an expression vector using a trp promoter, indole acrylic acid or the like may be added to the medium.

The expressed protein can be isolated and purified by a commonly used method. For example, in a case where the protein is expressed in a dissolved state in cells, the host cells are recovered by centrifugation after the completion of the culture, suspended in an aqueous buffer solution, and then disrupted using an ultrasonicator, a French press, a Manton-Gaulin homogenizer, a Dyno-Mill, or the like to obtain a cell-free extract. From the supernatant obtained by centrifuging the cell-free extract, a purified preparation can be obtained by a method commonly used for protein isolation and purification, that is, a solvent extraction method, a salting-out method using ammonium sulfate or the like, a desalting method, a precipitation method using an organic solvent, an anion exchange chromatography method using a resin such as diethylaminoethyl (DEAE)-Sepharose or DIAION HPA-75 (manufactured by Mitsubishi Kasei Kogyo Kabushiki Kaisha), an cation exchange chromatography method using a resin such as S-Sepharose FF (manufacture by Pharmacia Corporation), a hydrophobic chromatography method using a resin such as butyl sepharose or phenyl sepharose, a gel filtration method using a molecular sieve, an affinity chromatography method, a chromatofocusing method, or an electrophoresis method such as isoelectric focusing or the like, using the above methods singly or in combination thereof.

In addition, in a case where the protein is expressed to form an insoluble body in the cell, similarly, the host cells are recovered, disrupted and centrifuged to recover the insoluble body of the protein as a precipitated fraction. The recovered insoluble body of the protein can be solubilized with a protein denaturing agent. After this operation, a purified preparation of the protein can be obtained by the same isolation and purification method as described above. In a case where the protein is secreted extracellularly, the protein can be recovered from the culture supernatant. That is, a culture supernatant is obtained by treating the culture by a technique such as centrifugation, and a purified preparation can be obtained from the culture supernatant by using the same isolation and purification method as described above.

Hereinafter, each process of the production method for a protein molded article according to the present embodiment will be described in detail.

[Dissolving Process]

A dissolving process is a process of dissolving a protein in a solvent containing formic acid at a temperature of 40° C. or higher and lower than 80° C. to obtain a protein solution.

In the dissolving process, a purified protein may be used, or a protein in host cells expressing the protein (recombinant protein) may be used as the protein to be dissolved (hereinafter, also referred to as “target protein”). The purified protein may be a protein purified from host cells that have expressed the protein. In a case where a protein in the host cells is dissolved as the target protein, the host cells are brought into contact with a solvent containing formic acid to dissolve the protein in the host cells in the solvent containing formic acid. The host cells may be any cell that expresses the target protein and may be, for example, intact cells or cells that have been subjected to treatment such as disruption treatment. Alternatively, the cells may be cells subjected to a simple purification treatment in advance.

The method for purifying a protein from host cells that have expressed the protein is not particularly limited, but, for example, the methods disclosed in Japanese Patent No. 6077570 and Japanese Patent No. 6077569 can be used.

The solvent containing formic acid may be a solvent containing only formic acid or may be a mixed solvent containing other solvents in addition to formic acid. A commercially available product can be used for formic acid. Examples of the commercially available formic acid include formic acid manufactured by Wako Pure Chemical Industries, Ltd. The other solvent may be water.

In the solvent containing formic acid, the concentration of formic acid may be 30% by mass or more, 40% by mass or more, 50% by mass or more, and 60% by mass or more, 70% by mass or more, 80% by mass or more, 90% by mass or more, or 95% by mass or more, with respect to the total mass of the solvent. In the solvent containing formic acid, the concentration of formic acid may be 99% by mass or less, 95% by mass or less, 90% by mass or more, 80% by mass or less, 70% by mass or less, or 50% by mass or less, with respect to the total mass of the solvent.

The temperature (heating temperature) in the dissolving process is 40° C. or higher and lower than 80° C. and may be 40° C. or higher and 75° C. or lower, 50° C. or higher and 75° C. or lower, or 60° C. or higher and 75° C. or lower. The heating temperature may be lower than 80° C., 75° C. or lower, 70° C. or lower, 60° C. or lower, 50° C. or lower, or 40° C. or lower, and 40° C. or higher, 50° C. or higher, 60° C. or higher, or 65° C. or higher. In a case where the heating temperature in the dissolving process is high (40° C. or higher), the physical properties of a protein molded article are improved since the protein and the impurities that may be included in the protein can be further decomposed.

In the dissolving process, a protein may be dissolved in a solvent containing formic acid while maintaining the heating temperature described above. The time of maintaining the heating temperature, which is not particularly limited, may be 10 minutes or more and is preferably 10 to 120 minutes, more preferably 10 to 60 minutes, and still more preferably 10 to 30 minutes in consideration of industrial production. The time of maintaining the heating temperature may be appropriately set under the conditions that the protein is sufficiently dissolved but the impurities (other than the target protein) are less dissolved.

The addition amount of the solvent containing formic acid added to dissolve a protein is not particularly limited as long as it can dissolve the protein.

In a case of dissolving a purified protein, the addition amount of the solvent containing formic acid may be 1 to 100 times, 1 to 50 times, 1 to 25 times, 1 to 10 times, or 1 to 5 times with respect to the protein, as a rate (volume (mL)/weight (g)) of the volume (mL) of the solvent containing formic acid to the weight (g) of the protein (dry powder containing protein).

In a case of dissolving a protein in the host cells expressing the protein, the addition amount of the solvent containing formic acid may be 1 to 100 times, 1 to 50 times, 1 to 25 times, 1 to 10 times, or 1 to 5 times with respect to the protein, as a rate (volume (mL)/weight (g)) of the solvent containing formic acid (mL) to the weight (g) of the host cells.

The solvent containing formic acid may contain an inorganic salt. By adding an inorganic salt to the solvent containing formic acid, the solubility of protein can be increased.

Examples of the inorganic salt that can be added to the solvent containing formic acid include an alkali metal halide, an alkaline earth metal halide, an alkaline earth metal nitrate, a thiocyanate, and a perchlorate.

Examples of the alkali metal halide include potassium bromide, sodium bromide, lithium bromide, potassium chloride, sodium chloride, lithium chloride, sodium fluoride, potassium fluoride, cesium fluoride, potassium iodide, sodium iodide, and lithium iodide.

Examples of the alkaline earth metal halide include calcium chloride, magnesium chloride, magnesium bromide, calcium bromide, magnesium iodide, and calcium iodide.

Examples of the alkaline earth metal nitrate include calcium nitrate, magnesium nitrate, strontium nitrate, and barium nitrate.

Examples of thiocyanate include sodium thiocyanate, ammonium thiocyanate, and guanidinium thiocyanate.

Examples of perchlorate include ammonium perchlorate, potassium perchlorate, calcium perchlorate, silver perchlorate, sodium perchlorate, and magnesium perchlorate.

These inorganic salts may be used alone or in a combination of two or more thereof.

Suitable inorganic salts include an alkali metal halide and an alkaline earth metal halide. Specific examples of suitable inorganic salts include lithium chloride and calcium chloride.

The addition amount (content) of the inorganic salt may be 0.5% by mass or more and 10% by mass or less, or 0.5% by mass or more and 5% by mass or less, with respect to the total mass of the solvent containing formic acid.

The insoluble matter may be removed from the protein solution as necessary. That is, the production method for a protein molded article of the present embodiment may include a process of removing insoluble matter from the protein solution after the dissolving process, as necessary. Examples of the method for removing the insoluble matter from the protein solution include general methods such as centrifugation, and filter filtration with a drum filter, a press filter, or the like. In the case of filter filtration, the insoluble matter can be more efficiently removed from the protein solution by using a filter aid such as Celite or diatomaceous earth and a pre-coating agent in combination.

The protein solution contains a protein and a solvent containing formic acid that dissolves the protein (solvent for dissolving). The protein solution may contain impurities that may have been included together with the protein during the dissolving process. The protein solution may be a solution for molding a protein molded article.

The content of the protein in the protein solution may be 5% by mass or more and 35% by mass or less, or 5% by mass or more and 50% by mass or less with respect to the total amount of the protein solution.

As one embodiment of the present invention, a production method for a protein solution, which includes the above-described dissolving process, is provided. The production method for a protein solution according to the present embodiment includes a process of dissolving a protein in a solvent containing formic acid at a temperature of 40° C. or higher and lower than 80° C. to obtain a protein solution.

[Molding Process]

A molding process is a process of molding a protein molded article using a protein solution. The shape of the protein molded article is not particularly limited, but examples thereof include a fiber, a film, and a porous body.

In the protein solution, it is preferable to adjust the concentration and the viscosity of a protein, depending on the protein molded article to be molded.

The method for adjusting the concentration of a protein in the protein solution is not particularly limited, but, for example, a method for increasing the concentration of a protein by evaporating a solvent containing formic acid by distillation, or a method using a solution having a high concentration of a protein in the dissolving process, or a method for reducing the addition amount of a solvent containing formic acid with respect to the amount of a protein.

The viscosity suitable for spinning is generally 10 to 50,000 cP (centipoise), and the viscosity can be measured using, for example, an “EMS viscometer” (product name) manufactured by Kyoto Electronics Manufacturing Co., Ltd. In a case where the viscosity of the protein solution is not within the range of 10 to 10,000 cP (centipoise), the viscosity of the protein solution may be adjusted to a viscosity at which spinning can be performed. The viscosity can be adjusted using the methods described above or the like. The solvent containing formic acid may contain an inorganic salt as exemplified above.

In a case where the protein molded article to be molded is a protein fiber, the protein content (concentration) in the protein solution may be adjusted to the concentration and the viscosity at which spinning can be performed, as necessary. The method for adjusting the concentration and the viscosity of a protein is not particularly limited. As the spinning method, wet-type spinning and the like can be mentioned. In a case where a protein solution having the concentration and the viscosity suitable for spinning is added as a doping liquid to a coagulation liquid, the protein coagulates. At this time, since the protein solution is added to the coagulation liquid while maintaining a thread-shape, the protein coagulates in the thread shape and a yarn (undrawn yarn) can be formed. The undrawn yarn can be formed, for example, according to the method disclosed in Japanese Patent No. 5584932.

Wet-Type Spinning—Drawing

(a) Wet-Type Spinning

The coagulation liquid may be any solution that can be dissolved. As the coagulation liquid, it is preferable to use a lower alcohol having 1 to 5 carbon atoms such as methanol, ethanol, or 2-propanol, or acetone. The coagulation liquid may contain water. The temperature of the coagulation liquid is preferably 5 to 30° C. from the viewpoint of spinning stability.

The method for adding the protein solution while maintaining a thread-shape is not particularly limited, and examples thereof include a method for extruding the protein solution from a spinneret into a coagulation liquid in a desolvation bath. An undrawn yarn is obtained by coagulating the protein. The extrusion rate in a case of extruding the protein solution into the coagulation liquid can be appropriately set according to the diameter of the spinneret and the viscosity of the protein solution. For example, in a case of a syringe pump having a nozzle with a diameter of 0.1 to 0.6 mm, the extrusion rate is preferably 0.2 to 6.0 mL/h per hole, and more preferably 1.4 to 4.0 mL/h per hole from the viewpoint of spinning stability. The length of the desolvation bath (coagulation liquid bath) for containing the coagulation liquid is not particularly limited but may be, for example, 200 to 500 mm. The withdrawing speed of the undrawn yarn formed by coagulation of protein may be, for example, 1 to 14 m/min, and the residence time may be, for example, 0.01 to 0.15 min. The withdrawing speed of the undrawn yarn is preferably 1 to 3 m/min from the viewpoint of dissolution efficiency. The undrawn yarn formed by coagulation of protein may be further drawn (pre-drawn) in a coagulation liquid, but from the viewpoint of suppressing vaporization of a lower alcohol used in the coagulation liquid, it is preferable that the coagulation liquid is kept at a low temperature and the undrawn yarn is drawn from the coagulation liquid in the state of the undrawn yarn.

(b) Drawing

A process of further drawing the undrawn yarn obtained by the method described above may be included. The drawing may be one-stage drawing or multi-stage drawing including two or more stages. In a case where the drawing is performed in multiple stages, the molecules can be aligned in multiple stages and the total drawing rate can be increased, which is suitable for producing a fiber having high toughness.

In a case where the protein molded article to be molded is a film (a protein film), the protein solution may be adjusted to have the concentration and the viscosity that allow the protein solution to be formed into a film, as necessary. The method for forming a protein into a film is not particularly limited and includes a method in which a protein solution is applied to a flat plate having a resistance to a solvent containing formic acid to a predetermined thickness to form a coating film, and the solvent containing formic acid is removed from the plate, and then a film having a predetermined thickness is obtained.

As a method for forming a film having a predetermined thickness, for example, a casting method can be mentioned. In a case where a film is formed by a casting method, a protein film (a polypeptide film) can be obtained by casting, on a flat plate, the protein solution to a thickness of several microns or more using a device such as a doctor coat or a knife coater to form a cast film and subsequently removing the solvent by vacuum drying or immersion in a desolvation bath. The protein film can be formed according to the method disclosed in Japanese Patent No. 5678283.

In a case where the protein molded article to be molded is a porous body (a porous body of a protein), the protein solution may be adjusted to have the concentration and the viscosity that allow the protein solution to be formed into the porous body, as necessary. The method for forming a porous body of a protein is not particularly limited. For example, a method for obtaining a porous body by adding a proper amount of a foaming agent to a protein solution adjusted to have the concentration and the viscosity suitable for porosity and removing a solvent containing formic acid and the method disclosed in Japanese Patent No. 5796147 are mentioned.

[Production Method for Protein]

One embodiment of the present invention provides a production method for a protein, including: dissolving a target protein and impurities in a solvent containing formic acid at a temperature of 40° C. or higher and lower than 80° C. to obtain a protein solution containing the target protein; and treating the protein solution with a poor solvent for the target protein to aggregate the target protein, thereby obtaining the target protein as an aggregate. In the production method for a protein according to the present embodiment, a process of obtaining a protein solution containing the target protein may be carried out under the same conditions as in the dissolving process described above. The target protein may be the above-mentioned protein.

According to the production method for a protein of the present embodiment, most of the impurities are removed from the crude material containing the target protein to be purified and the impurities other than the target protein, and a purified target protein can be removed.

The target protein and impurities may be those extracted from a culture containing host cells that have produced the target protein by a gene recombination technique. The target protein and impurities may be those subjected to treatments such as centrifugation and filter filtration with respect to the target protein and impurities extracted from the culture containing host cells. In other words, the production method for a protein according to one embodiment may include: a process of causing host cells to produce a target protein in a culture; a process of obtaining a crude material containing the target protein and impurities from the culture; and a process of mixing the crude material with a solvent containing formic acid at a temperature of 40° C. or higher and lower than 80° C. to obtain a protein solution.

The poor solvent for the target protein is preferably a solvent that makes the target protein be hardly dissolved in the solvent contained in the protein solution. Examples of the poor solvent for the target protein include an aprotic polar solvent and a protic polar solvent.

Examples of the protic polar solvent may include water, methanol, ethanol, 1-propanol, 2-propanol (isopropanol), butanol, tert-butanol, ethylene glycol, propylene glycol, and glycerin.

Examples of the aprotic polar solvent include ketones and nitriles described later, N-methyl-2-pyrrolidone, dimethyl sulfoxide (DMSO), 1,3-dimethyl-2-imidazolidone (DMI), N,N-dimethylformamide (DMF), N,N-dimethylacetamide (DMA), propylene carbonate, hexamethylphosphoramide, N-ethylpyrrolidone, nitrobenzene, furfural, γ-butyrolactone, ethylene sulfite, sulfolane, and ethylene carbonate, but the examples are not limited thereto.

Examples of ketones include acetone, methyl ethyl ketone, methyl butyl ketone, and methyl isobutyl ketone.

The nitriles may be saturated or unsaturated, but saturated nitriles are preferred. The carbon number of the nitriles may be 2 to 8, preferably 2 to 6, and more preferably 2 to 4. Specific examples of the nitriles include acetonitrile, propionitrile, succinonitrile, butyronitrile, and isobutyronitrile.

The addition amount of the poor solvent for the target protein may be properly determined depending on the target protein so that the target protein precipitates. The poor solvent for the target protein may be added typically in the same amount as the protein solution. The addition amount may be properly adjusted depending on the state of aggregation of the target protein and the presence of impurities.

The poor solvent for the target protein may be a protic polar solvent or methanol from the viewpoint of further improving the purity of the target protein and the viewpoint of further increasing the recovery amount.

Examples of the method for recovering an aggregated target protein as the aggregate include general methods such as centrifugation, and filter filtration with a drum filter, a press filter, or the like. In the case of filter filtration, a target protein of interest can be more efficiently recovered as the aggregate by using a filter aid such as Celite or diatomaceous earth and a pre-coating agent in combination.

### EXAMPLES

Hereinafter, the present invention will be described more specifically based on Examples. However, the present invention is not limited to the following Examples.

[(1) Preparation Strain (Recombinant Cell) Expressing Target Protein]

A nucleic acid encoding a spider silk fibroin (PRT775) having the amino acid sequence set forth in SEQ ID NO: 46 was synthesized. In the nucleic acid, an NdeI site was added to the 5′ end and an EcoRI site was added downstream of the stop codon. The hydropathy index and the molecular weight of each protein are as shown in Table 4.

TABLE 4

| Sequence ID No. | Protein name | Hydropathy index | Molecular weight |
|---|---|---|---|
| 46 | PRT775 | −0.59 | 99.7 |

In the same manner as described above, a nucleic acid encoding a spider silk fibroin (PRT799) having the amino acid sequence set forth in SEQ ID NO: 15 and a nucleic acid encoding a spider silk fibroin (PRT918) having the amino acid sequence set forth in SEQ ID NO: 39 were synthesized. In the nucleic acid, an NdeI site was added to the 5′ end and an EcoRI site was added downstream of the stop codon.

Each of the above nucleic acids was cloned into a cloning vector (pUC118). Thereafter, the nucleic acid was enzymatically cleaved by treatment with NdeI and EcoRI and then recombinated into a protein expression vector pET-22b(+) to obtain an expression vector. Escherichia coli BLR(DE3) was transformed with the pET-22b (+) expression vector in which the nucleic acid had been recombined, to obtain a transformed Escherichia coli (recombinant cell) expressing the target protein.

[(2) Expression of Target Protein]

The above transformed Escherichia coli was cultured in 2 mL of an LB medium containing ampicillin for 15 hours. The culture solution was added to 100 mL of a seed culture medium (Table 5) containing ampicillin so that the OD₆₀₀ was 0.005. While maintaining the temperature of the culture solution at 30° C., flask culturing was carried out (for about 15 hours) until the OD₆₀₀ reached 5, thereby obtaining a seed culture solution.

TABLE 5
Seed culture medium

| Reagent | Concentration (g/L) |
|---|---|
| Glucose | 5.0 |
| KH₂PO₄ | 4.0 |
| K₂HPO₄ | 10.0 |
| Yeast Extract | 6.0 |
| Ampicillin | 0.1 |

The seed culture solution was added to a jar fermenter containing 500 mL of a production medium (Table 6) so that the OD₆₀₀ was 0.05. The culture was carried out while keeping the culture solution temperature at 37° C. and controlling the pH constant at 6.9. Further, the concentration of dissolved oxygen in the culture solution was maintained at 20% of the dissolved oxygen saturation concentration.

TABLE 6
Production medium

| Reagent | Concentration (g/L) |
|---|---|
| Glucose | 12.0 |
| KH₂PO₄ | 9.0 |
| MgSO₄•7H₂0 | 2.4 |
| Yeast Extract | 15 |
| FeSO₄•7H₂0 | 0.04 |
| MnSO₄ 5H₂0 | 0.04 |
| CaCl₂•2H₂0 | 0.04 |
| GD-113 (anti-foaming agent) | 0.1 (mL/L) |

Immediately after glucose in the production medium was completely consumed, a feed solution (455 g/lL of glucose and 120 g/lL of Yeast Extract) was added at a rate of 1 mL/min. The culture was carried out while keeping the culture solution temperature at 37° C. and controlling the pH constant at 6.9. The culture was carried out for 20 hours while the concentration of dissolved oxygen in the culture solution was maintained at 20% of the dissolved oxygen saturation concentration. Thereafter, 1 M isopropyl-β-thiogalactopyranoside (IPTG) was added to the culture solution to a final concentration of 1 mM to induce the expression of the target protein. 20 hours after the addition of IPTG, the culture solution was centrifuged to recover the wet bacterial cells. SDS-PAGE was carried out using bacterial cells (wet bacterial cells) prepared from the culture solution before the addition of IPTG and after the addition of IPTG and the expression of the target protein as an insoluble body was checked by the IPTG addition-dependent appearance of a band equivalent to a target protein size. The recovered wet bacterial cells were dried to obtain dry bacterial cells of Escherichia coli expressing a spider silk fibroin. By the above operations, wet bacterial cells and dry bacterial cells each expressing each of spider silk fibroins PRT775, PRT799, and PRT918 were obtained.

[(3) Purification of Protein (PRT775)]

The bacterial cells expressing PRT775 recovered 2 hours after the addition of IPTG were washed with a 20 mM Tris-HCl buffer solution (pH 7.4). The bacterial cells after washing were suspended in 20 mM Tris-HCl buffer solution (pH 7.4) containing about 1 mM PMSF, and the cell suspension was disrupted with a high-pressure homogenizer (available from GEA Niro Soavi SpA). The disrupted cells were centrifuged to obtain a precipitate. The obtained precipitate was washed with a 20 mM Tris-HCl buffer solution (pH 7.4) until the obtained precipitate was highly pure. The precipitate after washing was suspended in 8 M guanidine buffer solution (8 M guanidine hydrochloride, 10 mM sodium dihydrogen phosphate, 20 mM NaCl, 1 mM Tris-HCl, pH 7.0) so that the concentration of the suspension was 100 mg/mL, and dissolved by stirring with a stirrer at 60° C. for 30 minutes. After dissolving, dialysis was carried out in water using a dialysis tube (cellulose tube 36/32 manufactured by Sanko Junyaku Co., Ltd.). The white protein aggregate obtained after dialysis was recovered by centrifugation, the water content was removed with a lyophilizer, and a lyophilized powdery protein (PRT775) was recovered.

[(4) Preparation of Doping Liquid]

1. A predetermined amount of formic acid was weighed in a screw tube bottle made of Pyrex (registered trademark) glass.

2. A purified target protein (PRT775) was weighed such that the protein content was 25% by mass when dissolved, and put into the screw tube bottle.

3. Samples (1 to 6) containing the target protein and formic acid obtained in “2” was stirred at a predetermined temperature for 3 hours or more using a stirrer (the heating temperatures of the samples 1 to 6 were respectively room temperature (RT, 25° C.), 70° C., 40° C., 50 C, 60° C., and 80° C.).

4. Each sample was degassed for 30 minutes or longer with Awatori Kentaro (ARE-500) to obtain a doping liquid.

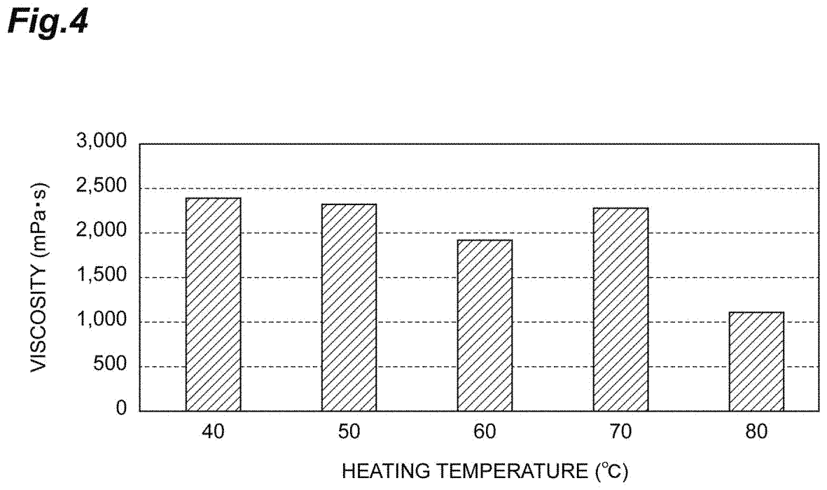

5. Each doping liquid obtained in “4” was dispensed into a test tube for measuring viscosity, and the viscosity was measured. Table 7 shows the evaluation results of the determination of dissolution and the measurement results of the viscosity. FIG. 4 shows the measurement results of the viscosity at each heating temperature. The solubility was visually evaluated. In the determination of dissolution, “B” indicates that it is insoluble, and “A” indicates that it is dissolved. The viscosity was measured using an “EMS viscometer” (product name) manufactured by Kyoto Electronics Manufacturing Co., Ltd.

TABLE 7

| | | Determination | Viscosity (mPa · s) | | |
|---|---|---|---|---|---|
| Sample | Temperature | of dissolution | 20° C. | 30° C. | 40° C. |
| 1 | RT | B | — | — | — |
| 2 | 40° C. | A | 5,030 | 3,280 | 2,390 |
| 3 | 50° C. | A | 4,870 | 3,060 | 2,320 |
| 4 | 60° C. | A | 4,180 | 2,650 | 1,920 |
| 5 | 70° C. | A | 5,050 | 3,290 | 2,280 |
| 6 | 80° C. | A | 2,300 | 1,520 | 1,110 |

[(5) Molding of Protein Fiber]

Each doping liquid (protein concentration in the doping liquid: 25% by mass) was discharged by a gear pump into a coagultination liquid (methanol) using a known spinning device. The spinning conditions were as shown below. As a result, a protein fiber (a fibroin fiber) was obtained as a protein molded article.

(Spinning Conditions)

Temperature of doping liquid: 25° C.

Hot roller (HR) temperature: 60° C.

Total drawing rate: 4 times (sample 1), 5 times (samples 2 to 5), 1 time (sample 6)

[(6) Tensile Test of Protein Fiber]

The protein fiber was fixed on a test paper piece by an adhesive agent with a distance between gripping jigs of 20 mm, and stress (strength) and elongatability were measured at a tensile speed of 10 cm/min using a tensile tester 3342 manufactured by Illinois Tool Works Inc. under the conditions of a temperature of 20° C. and a relative humidity of 65%. The load cell capacity was 10 N, and the gripping jig was a clip type.

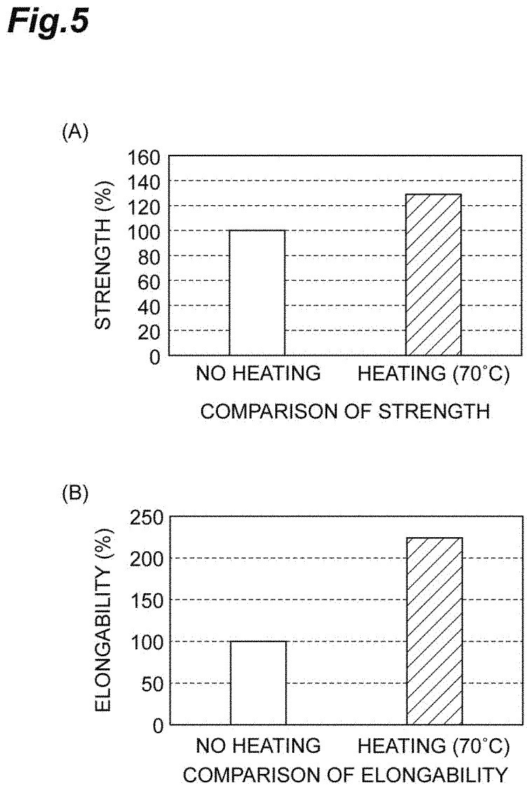

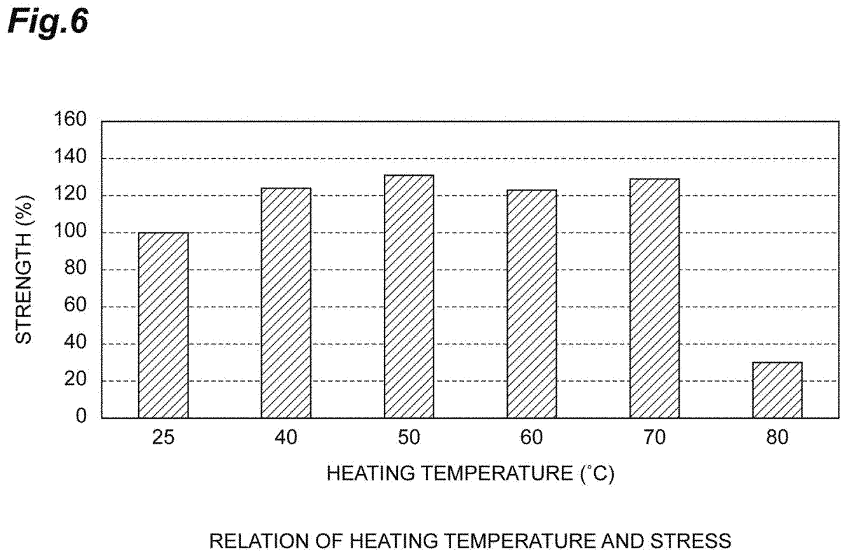

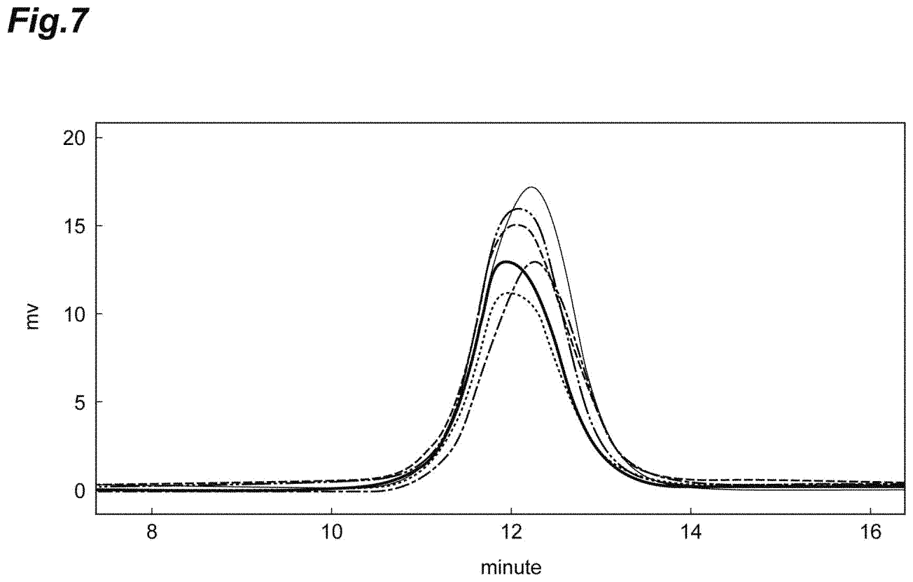

The results are shown in FIGS. 5 to 6 and Table 8. Table 8 shows the values of strength (MPa) and elongatability (%) (average value of sample number n=10). FIG. 7 shows the GPC measurement results of each sample (1 to 6). In FIG. 7, the solid line (thin) indicates a result obtained with a heating (70° C.), the two-dotted chain line indicates a result obtained with a heating (60° C.), the broken line indicates a result obtained without a heating (25° C.), the one-dotted chain line indicates a result obtained with a heating (80° C.), the solid line (thick) indicates a result obtained with a heating (40° C.), and the dotted line indicates a result obtained with a heating (50° C.).

TABLE 8

| Sample | Strength (%) | Elongability (%) | Note |
|---|---|---|---|
| 1 | 100 | 100 | No heating |
| 2 | 124 | 249 | Heating (40° C.) |
| 3 | 131 | 271 | Heating (50° C.) |
| 4 | 123 | 255 | Heating (60° C.) |
| 5 | 129 | 224 | Heating (70° C.) |
| 6 | 30 | 16 | Heating (80° C.) |

### Test Example 1

### Production of Target Protein PRT799 Using Wet Bacterial Cells

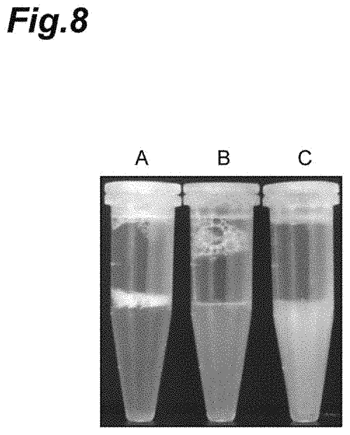

Wet bacterial cells containing a spider silk fibroin PRT799 were aliquoted to a volume of 500 μL and centrifuged at 2,500 g for 10 minutes. The supernatant obtained after centrifugation was removed from the sample, and then the same amount as that of the supernatant removed, which was formic acid or a mixed solvent (formic acid aqueous solution) of formic acid and water (reverse osmosis membrane (RO) water) was added to the sample. The concentration of formic acid in the formic acid aqueous solution was 75% by mass or 50% by mass with respect to the total amount of the formic acid aqueous solution. The sample to which the above solvent was added was stirred for 1 hour under the condition of 1,500 rpm while being heated to 40° C. to obtain a protein solution. A photograph of the obtained protein solutions is shown in FIG. 8. FIG. 8A shows a protein solution obtained by adding formic acid to wet bacterial cells, FIG. 8B shows a protein solution obtained by adding a 75% by mass formic acid aqueous solution to wet bacterial cells, and FIG. 8C shows a protein solution obtained by adding a 50% by mass formic acid aqueous solution to wet bacterial cells.

Each protein solution was centrifuged for 10 minutes at 2,500 g. The supernatant obtained after centrifugation was added to 1.5 times the volume of methanol and allowed to be left for 2 hours. After allowing to be left for 2 hours, centrifugation was performed at 2,500 g for 10 minutes, the supernatant was removed, and washing was performed twice with an equal volume of RO water. After the washing was completed, the precipitate was lyophilized to recover a powdery sample containing protein.

The amount of total protein and the amount of fibroin in the obtained sample were measured. The total protein amount was measured by the BCA method. The amount of fibroin was measured using a Ni sepharose. The purity of fibroin is denoted by the proportion (fibroin amount/total protein amount×100) of the amount of fibroin (mg/mL) to the amount of total protein (mg/mL). The results of the purity of fibroin and the recovered amount are shown in Table below.

TABLE 9

| Sample | Purity of protein (%) | Recovered amount (mg) |
|---|---|---|
| Formic acid 100% | 61.2 | 9.0 |
| Formic acid 75% | 59.1 | 8.6 |
| Formic acid 50% | 64.8 | 3.3 |

(Analysis by SDS-PAGE)

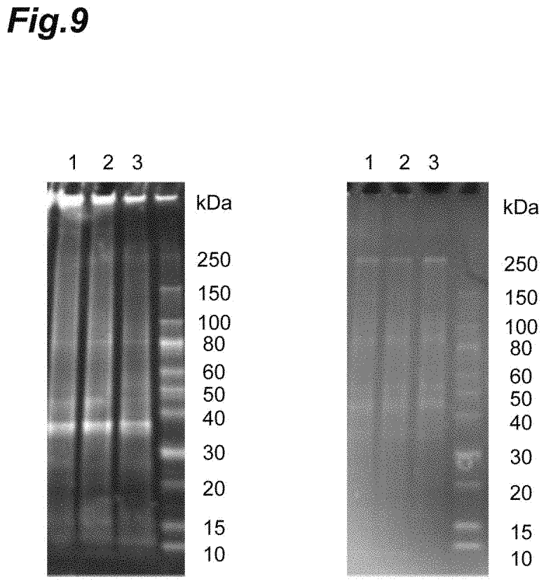

Each sample was analyzed by SDS-PAGE. In the analysis result of FIG. 9, lane No. 1 shows a result obtained in a case of formic acid, lane No. 2 is a result obtained in a case of 75% by mass formic acid aqueous solution, and Lane No. 3 is a result obtained in a case of 50% by mass formic acid aqueous solution.

With respect to each of the powder containing the spider silk fibroin, an SDS-PAGE sample was prepared such that the protein concentration was 10 mg/mL, based on the result obtained by the measurement by the BCA method (Mini-PROTEAN (registered trademark) TetraSystem and Mini-PROTEAN (registered trademark)).

TGX (registered trademark) Gels was set, each of the prepared SDS-PAGE samples was loaded, and electrophoresis was performed. After the electrophoresis was completed, the analysis was performed with GelDOC (registered trademark) EZImager. The results are shown in FIG. 9.

The photograph on the left of FIG. 9 is a photograph after electrophoresis, obtained by staining with an Oriole (trademark) fluorescent gel stain (Bio-Rad Laboratories, Inc.) that can stain all proteins, and the photograph on the right of FIG. 1 is a photograph after electrophoresis, obtained by staining with an InVision (trademark) His-tag in-gel staining reagent (ThermoFisher Scientific Co., Ltd.) that reacts to the His-tag region of PRT799. PRT799 (theoretical molecular weight: 211.4 kDa) was detected as a band near the molecular weight marker of 250 kDa.

### Test Example 2

### Production (1) of Target Protein (PRT799) Using Dry Bacterial Cells

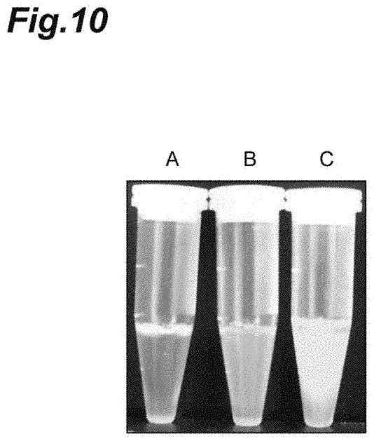

500 μL of formic acid or a mixed solvent (formic acid aqueous solution) of formic acid and water (reverse osmosis membrane (RO) water) was added to 50 mg of dry bacterial cells containing a spider silk fibroin PRT799. The concentration of formic acid in the formic acid aqueous solution was 75% by mass or 50% by mass with respect to the total amount of the formic acid aqueous solution. The sample to which the above solvent was added was stirred for 1 hour under the condition of 1,500 rpm while being heated to 40° C. to obtain a protein solution. A photograph of the obtained protein solutions is shown in FIG. 10. FIG. 10A shows a protein solution obtained by adding formic acid to dry bacterial cells, FIG. 10B shows a protein solution obtained by adding a 75% by mass formic acid aqueous solution to dry bacterial cells, and FIG. 10C shows a protein solution obtained by adding a 50% by mass formic acid aqueous solution to dry bacterial cells.

Using the obtained protein solution, the same operation as in Test Example 1 was performed to obtain a powdery sample. The total protein amount and the fibroin amount in the obtained sample were measured in the same manner as in Test Example 1. The results of the purity of fibroin and the recovered amount are shown in Table below.

TABLE 10

| Sample | Purity of protein (%) | Recovered amount (mg) |
|---|---|---|
| Formic acid 100% | 54.8 | 11.6 |
| Formic acid 75% | 51.9 | 11.7 |
| Formic acid 50% | 58.6 | 7.6 |

(Analysis by SDS-PAGE)

The obtained sample containing a spider fibroin was analyzed by the same method as described in “Analysis by SDS-PAGE”. In the analysis result of FIG. 10, lane No. 1 shows a result obtained in a case of formic acid, lane No. 2 is a result obtained in a case of 75% by mass formic acid aqueous solution, and Lane No. 3 is a result obtained in a case of 50% by mass formic acid aqueous solution.

### Test Example 3

### Production (2) of Target Protein PRT799 Using Dry Bacterial Cells

100 mL of a mixed solvent (formic acid aqueous solution) of formic acid and water (reverse osmosis membrane (RO) water) was added to 10 g of dry bacterial cells containing a spider silk fibroin PRT799. The concentration of formic acid in the formic acid aqueous solution was 50% by mass with respect to the total amount of the formic acid aqueous solution. The sample to which the above solvent was added was stirred for 1 hour under the condition of 1,500 rpm while being heated to 40° C. to obtain a protein solution. 10 g of a filtration aid was added to the protein solution, and suction filtration was performed using a disposable bottle top filter pre-coated with the filtration aid. The supernatant obtained after suction filtration was added to 1.5 times the volume of methanol and allowed to be left for 2 hours. After allowing to be left for 2 hours, centrifugation was performed at 2,500 g for 10 minutes, the supernatant was removed, and washing was performed twice with an equal volume of RO water. After the washing was completed, the precipitate was lyophilized to recover a powdery sample containing protein.

(Analysis by SDS-PAGE)

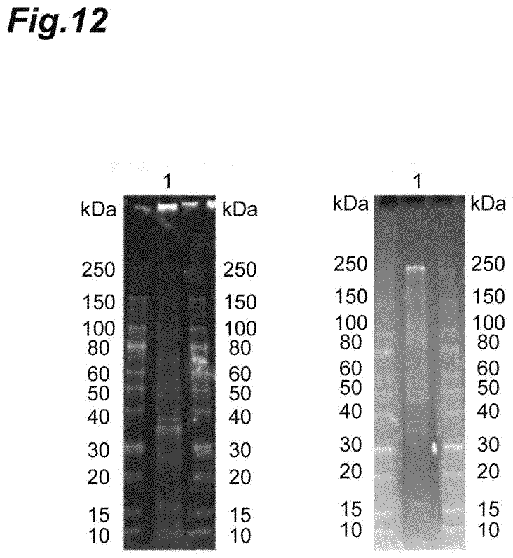

The obtained sample was analyzed by the same method as described in “Analysis by SDS-PAGE”. The analysis result is shown in lane No. 1 in FIG. 12.

### Test Example 4

### Production of Target Protein PRT918 Using Dry Bacterial Cells

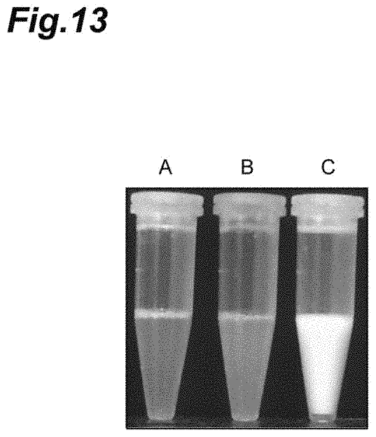

500 μL of formic acid or a mixed solvent (formic acid aqueous solution) of formic acid and water (reverse osmosis membrane (RO) water) was added to 50 mg of dry bacterial cells containing a spider silk fibroin PRT918. The concentration of formic acid in the formic acid aqueous solution was 75% by mass or 50% by mass with respect to the total amount of the formic acid aqueous solution. The sample to which the above solvent was added was stirred for 1 hour under the condition of 1,500 rpm while being heated to 40° C. to obtain a protein solution. A photograph of the obtained protein solutions is shown in FIG. 13. FIG. 13A shows a protein solution obtained by adding formic acid to dry bacterial cells, FIG. 13B shows a protein solution obtained by adding a 75% by mass formic acid aqueous solution to dry bacterial cells, and FIG. 13C shows a protein solution obtained by adding a 50% by mass formic acid aqueous solution to dry bacterial cells.

Each protein solution was centrifuged for 10 minutes at 2,500 g. The supernatant obtained after centrifugation was added to 2 times the volume of RO water and allowed to be left for 2 hours. After allowing to be left for 2 hours, centrifugation was performed at 2,500 g for 10 minutes, the supernatant was removed, and washing was performed twice with an equal volume of RO water. After the washing was completed, the precipitate was lyophilized to recover a powdery sample containing protein.

(Analysis by SDS-PAGE)

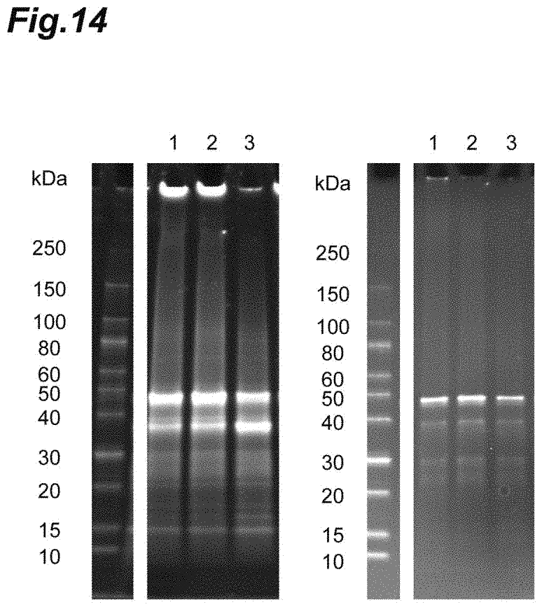

The obtained sample containing a spider fibroin was analyzed by the same method as described in “Analysis by SDS-PAGE”. In the analysis result of FIG. 14, lane No. 1 shows a result obtained in a case of formic acid, lane No. 2 is a result obtained in a case of 75% by mass formic acid aqueous solution, and Lane No. 3 is a result obtained in a case of 50% by mass formic acid aqueous solution.

1. A production method for a protein molded article, comprising: dissolving a protein in a solvent containing formic acid at a temperature of 40° C. or higher and lower than 80° C. to obtain a protein solution; and
molding a protein molded article using the protein solution.

2. The production method for a protein molded article according to claim 1, wherein the protein molded article is a protein fiber.

3. The production method for a protein molded article according to claim 1, wherein the protein is a structural protein.

4. The production method for a protein molded article according to claim 3, wherein the structural protein is a spider silk fibroin.

5. A production method for a protein solution, comprising:
dissolving a protein in a solvent containing formic acid at a temperature of 40° C. or higher and lower than 80° C. to obtain a protein solution.

6. The production method according to claim 5, wherein the protein is a structural protein.

7. The production method for according to claim 6, wherein the structural protein is a spider silk fibroin.

8. A production method for a protein, comprising:
dissolving a target protein and impurities in a solvent containing formic acid at a temperature of 40° C. or higher and lower than 80° C. to obtain a protein solution containing the target protein; and
treating the protein solution with a poor solvent for the target protein to aggregate the target protein, thereby obtaining the target protein as an aggregate.


SEQUENCE LISTING (46 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 1154 aa, Artificial Sequence
  recombinant spider silk protein ADF3KaiLargeNRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 5 - protein, 24 aa, Artificial Sequence
  His tag and start codon
MHHHHHHHHHHSSGSSLEVLFQGP

SEQ ID NO: 6 - protein, 597 aa, Artificial Sequence
  Met-PRT380
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 7 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 8 - protein, 565 aa, Artificial Sequence
  Met-PRT468
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 9 - protein, 2364 aa, Artificial Sequence
  Met-PRT799
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 10 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 11 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 12 - protein, 12 aa, Artificial Sequence
  HisTag
MHHHHHHSSGSS

SEQ ID NO: 13 - protein, 608 aa, Artificial Sequence
  PRT380
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 14 - protein, 576 aa, Artificial Sequence
  PRT468
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 15 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 16 - protein, 608 aa, Artificial Sequence
  PRT313
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 17 - protein, 601 aa, Artificial Sequence
  PRT399
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 18 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 19 - protein, 612 aa, Artificial Sequence
  Met-PRT720
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 20 - protein, 592 aa, Artificial Sequence
  Met-PRT665
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 21 - protein, 619 aa, Artificial Sequence
  Met-PRT666
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 22 - protein, 590 aa, Artificial Sequence; identical to SEQ ID NO: 7
  Met-PRT410

SEQ ID NO: 23 - protein, 565 aa, Artificial Sequence; identical to SEQ ID NO: 8
  Met-PRT468

SEQ ID NO: 24 - protein, 623 aa, Artificial Sequence
  PRT468
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 25 - protein, 603 aa, Artificial Sequence
  PRT665
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 26 - protein, 630 aa, Artificial Sequence
  PRT666
MHHHHHHSSGSSGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 27 - protein, 593 aa, Artificial Sequence
  M_PRT888
MGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 28 - protein, 590 aa, Artificial Sequence
  M_PRT965
MGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 29 - protein, 593 aa, Artificial Sequence
  M_PRT889
MGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 30 - protein, 590 aa, Artificial Sequence
  M_PRT916
MGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 31 - protein, 590 aa, Artificial Sequence
  M_ PRT918
MGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 32 - protein, 565 aa, Artificial Sequence
  M_PRT699
MGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 33 - protein, 565 aa, Artificial Sequence
  M_PRT698
MGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 34 - protein, 565 aa, Artificial Sequence; identical to SEQ ID NO: 8
  M_PRT525

SEQ ID NO: 35 - protein, 601 aa, Artificial Sequence
  PRT888
MHHHHHHSSGSSGPGVLGPYGPGASAAAAAGQNGPGSGVLGPGQSGQYGPGVLGPGVLGPGSSAAAAAGPGQYGPGVLGPSASAAAAAGPGSGVLGPGASGQYGPGVLGPGVLGPGSSAAAAAGQYGSGPGVLGPYGSAAAAAGPGSGQYGQGPYGPGASGPGQYGPGVLGPSASAAAAAGSGVLGPGQYGPYASAAAAAGQYGSGPGVLGPYGPGQSGSGVLGPGVLGPYASAAAAAGPGVLGPYGPGSSAAAAAGQYGYGPGVLGPYGPGASGQNGPGSGQYGPGVLGPGQSAAAAAGPGVLGPYGPGASAAAAAGQYGPGVLGPGQYGPGSSGPGVLGPYGPGSSAAAAAGQYGPGVLGPYGPGQSAAAAAGQYVLGPGVLGPYGPGASGPGVLGPYGPGASAAAAAGPGQYGPGVLGPSASAAAAAGQYGSGPGQYGPYGPGQSGPGSGVLGQGPYGPGASAAAAAGQYGPGVLGPYGPGQSAAAAAGPGSGQYGPGASGQNGPGSGQYGPGVLGPGQSAAAAAGQYVLGPGVLGPYGPGASAAAAAGQYGSGPGVLGPYGPGQSGSGVLGPGVLGPYASAAAAAGPGSGVLGPGAS

SEQ ID NO: 36 - protein, 601 aa, Artificial Sequence
  PRT965
MHHHHHHSSGSSGPGTSGPYGPGASAAAAAGANGPGSGTSGPGASGAYGPGTSGPGTSGPGSSAAAAAGPGAYGPGTSGPSASAAAAAGPGSGTSGPGASGAYGPGTSGPGTSGPGSSAAAAAGAYGSGPGTSGPYGSAAAAAGPGSGAYGAGPYGPGASGPGAYGPGTSGPSASAAAAAGSGTSGPGAYGPYASAAAAAGAYGSGPGTSGPYGPGASGSGTSGPGTSGPYASAAAAAGPGTSGPYGPGSSAAAAAGAYGYGPGTSGPYGPGASGANGPGSGAYGPGTSGPGASAAAAAGPGTSGPYGPGASAAAAAGAYGPGTSGPGAYGPGSSGPGTSGPYGPGSSAAAAAGAYGPGTSGPYGPGASAAAAAGAYTSGPGTSGPYGPGASGPGTSGPYGPGASAAAAAGPGAYGPGTSGPSASAAAAAGAYGSGPGAYGPYGPGASGPGSGTSGAGPYGPGASAAAAAGAYGPGTSGPYGPGASAAAAAGPGSGAYGPGASGANGPGSGAYGPGTSGPGASAAAAAGAYTSGPGTSGPYGPGASAAAAAGAYGSGPGTSGPYGPGASGSGTSGPGTSGPYASAAAAAGPGSGTSGPGAS

SEQ ID NO: 37 - protein, 601 aa, Artificial Sequence
  PRT889
MHHHHHHSSGSSGPGVLGPYGPGASAAAAAGINGPGSGVLGPGISGIYGPGVLGPGVLGPGSSAAAAAGPGIYGPGVLGPSASAAAAAGPGSGVLGPGASGIYGPGVLGPGVLGPGSSAAAAAGIYGSGPGVLGPYGSAAAAAGPGSGIYGIGPYGPGASGPGIYGPGVLGPSASAAAAAGSGVLGPGIYGPYASAAAAAGIYGSGPGVLGPYGPGISGSGVLGPGVLGPYASAAAAAGPGVLGPYGPGSSAAAAAGIYGYGPGVLGPYGPGASGINGPGSGIYGPGVLGPGISAAAAAGPGVLGPYGPGASAAAAAGIYGPGVLGPGIYGPGSSGPGVLGPYGPGSSAAAAAGIYGPGVLGPYGPGISAAAAAGIYVLGPGVLGPYGPGASGPGVLGPYGPGASAAAAAGPGIYGPGVLGPSASAAAAAGIYGSGPGIYGPYGPGISGPGSGVLGIGPYGPGASAAAAAGIYGPGVLGPYGPGISAAAAAGPGSGIYGPGASGINGPGSGIYGPGVLGPGISAAAAAGIYVLGPGVLGPYGPGASAAAAAGIYGSGPGVLGPYGPGISGSGVLGPGVLGPYASAAAAAGPGSGVLGPGAS

SEQ ID NO: 38 - protein, 601 aa, Artificial Sequence
  PRT916
MHHHHHHSSGSSGPGVIGPYGPGASAAAAAGLNGPGSGVIGPGLSGLYGPGVIGPGVIGPGSSAAAAAGPGLYGPGVIGPSASAAAAAGPGSGVIGPGASGLYGPGVIGPGVIGPGSSAAAAAGLYGSGPGVIGPYGSAAAAAGPGSGLYGLGPYGPGASGPGLYGPGVIGPSASAAAAAGSGVIGPGLYGPYASAAAAAGLYGSGPGVIGPYGPGLSGSGVIGPGVIGPYASAAAAAGPGVIGPYGPGSSAAAAAGLYGYGPGVIGPYGPGASGLNGPGSGLYGPGVIGPGLSAAAAAGPGVIGPYGPGASAAAAAGLYGPGVIGPGLYGPGSSGPGVIGPYGPGSSAAAAAGLYGPGVIGPYGPGLSAAAAAGLYVIGPGVIGPYGPGASGPGVIGPYGPGASAAAAAGPGLYGPGVIGPSASAAAAAGLYGSGPGLYGPYGPGLSGPGSGVIGLGPYGPGASAAAAAGLYGPGVIGPYGPGLSAAAAAGPGSGLYGPGASGLNGPGSGLYGPGVIGPGLSAAAAAGLYVIGPGVIGPYGPGASAAAAAGLYGSGPGVIGPYGPGLSGSGVIGPGVIGPYASAAAAAGPGSGVIGPGAS

SEQ ID NO: 39 - protein, 601 aa, Artificial Sequence
  PRT918
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS

SEQ ID NO: 40 - protein, 576 aa, Artificial Sequence
  PRT699
MHHHHHHSSGSSGPGVLGPYGPGASAAAAAAAGSNGPGSGVLGPGQSGQYGPGVLGPGVLGPGSSAAAAAAAGPGQYGPGVLGPSASAAAAAAAGPGSGVLGPGASGQYGPGVLGPGVLGPGSSAAAAAAAGSYGSGPGVLGPYGSAAAAAAAGPGSGQYGQGPYGPGASGPGQYGPGVLGPSASAAAAAAAGSGVLGPGQYGPYASAAAAAAAGSYGSGPGVLGPYGPGQSGSGVLGPGVLGPYASAAAAAAAGPGVLGPYGPGSSAAAAAAAGSYGYGPGVLGPYGPGASGQNGPGSGQYGPGVLGPGPSAAAAAAAGPGVLGPYGPGASAAAAAAAGSYGPGVLGPGQYGPGSSGPGVLGPYGPGSSAAAAAAAGSYGPGVLGPYGPGPSAAAAAAAGSYVLGPGVLGPYGPGASGPGVLGPYGPGASAAAAAAAGPGQYGPGVLGPSASAAAAAAAGSYGSGPGQYGPYGPGQSGPGSGVLGQGPYGPGASAAAAAAAGSYGPGVLGPYGPGPSAAAAAAAGPGSGQYGPGASGQNGPGSGQYGPGVLGPGPSAAAAAAAGPGSGVLGPGAS

SEQ ID NO: 41 - protein, 576 aa, Artificial Sequence
  PRT698
MHHHHHHSSGSSGPGVLGPYGPGASAAAAAAAGSNGPGSGVLGPGISGIYGPGVLGPGVLGPGSSAAAAAAAGPGIYGPGVLGPSASAAAAAAAGPGSGVLGPGASGIYGPGVLGPGVLGPGSSAAAAAAAGSYGSGPGVLGPYGSAAAAAAAGPGSGIYGIGPYGPGASGPGIYGPGVLGPSASAAAAAAAGSGVLGPGIYGPYASAAAAAAAGSYGSGPGVLGPYGPGISGSGVLGPGVLGPYASAAAAAAAGPGVLGPYGPGSSAAAAAAAGSYGYGPGVLGPYGPGASGINGPGSGIYGPGVLGPGPSAAAAAAAGPGVLGPYGPGASAAAAAAAGSYGPGVLGPGIYGPGSSGPGVLGPYGPGSSAAAAAAAGSYGPGVLGPYGPGPSAAAAAAAGSYVLGPGVLGPYGPGASGPGVLGPYGPGASAAAAAAAGPGIYGPGVLGPSASAAAAAAAGSYGSGPGIYGPYGPGISGPGSGVLGIGPYGPGASAAAAAAAGSYGPGVLGPYGPGPSAAAAAAAGPGSGIYGPGASGINGPGSGIYGPGVLGPGPSAAAAAAAGPGSGVLGPGAS

SEQ ID NO: 42 - protein, 252 aa, Artificial Sequence
  Collagen-type4-Kai
MHHHHHHSSGSSKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEK

SEQ ID NO: 43 - protein, 310 aa, Artificial Sequence
  Resilin-Kai
MHHHHHHPEPPVNSYLPPSDSYGAPGQSGPGGRPSDSYGAPGGGNGGRPSDSYGAPGQGQGQGQGQGGYAGKPSDSYGAPGGGDGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASG

SEQ ID NO: 44 - protein, 282 aa, Artificial Sequence
  elastin short
MHHHHHHSSGSSLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVG

SEQ ID NO: 45 - protein, 468 aa, Artificial Sequence
  type I keratin 26
MSFRLSGVSRRLCSQAGTGRLTGGRTGFRAGNVCSGLGAGSSFSGPLGSVSSKGSFSHGGGGLGSGVCTGFLENEHGLLPGNEKVTLQNLNDRLASYLDHVCTLEEANADLEQKIKGWYEKYGPGSGRQLAHDYSKYFSVTEDLKRQIISVTTCNASIVLQNENARLTADDFRLKCENELALHQSVEADINGLHRVMDELTLCTSDLEMQCEALSEELTYLKKNHQEEMKVMQGAARGNVNVEINAAPGVDLTVLLNNMRAEYEDLAEQNHEDAEAWFSEKSTSLHQQISDDAGAAMAARNELMELKRNLQTLEIELQSLLAMKHSYECSLAETESNYCHQLQQIQEQIGAMEDQLQQIRMETEGQKLEHERLLDVKIFLEKEIEMYCKLIDGEGRKSKSTCYKSEGRGPKNSENQVKDSKEEAVVKTVVGELDQLGSVLSLRVHSVEEKSSKISNITMEQRLPSKVP

SEQ ID NO: 46 - protein, 1146 aa, Artificial Sequence
  PRT775
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGASELGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGASHHHHHH